(19) United States
(12) Patent Application Publication
(10) Pub. No.: US 2018/0009825 A1
(43) Pub. Date: Jan. 11, 2018

(54) METHODS OF MAKING ERIBULIN MESYLATE

(71) Applicant: Apicore US LLC, Somerset, NJ (US)

(72) Inventors: Ravishanker Kovi, Monroe, NJ (US); Sanjay Thakor, Gujrat (IN); Ashish Naik, Piscataway, NJ (US); Jayaraman Kannapan, Gujarat (IN)

(73) Assignee: Apicore US LLC, Somerset, NJ (US)

(21) Appl. No.: 15/641,831

(22) Filed: Jul. 5, 2017

Related U.S. Application Data
(60) Provisional application No. 62/358,921, filed on Jul. 6, 2016.

(51) Int. Cl. C07D 493/22; C07F 7/18

(52) U.S. Cl. CPC C07D 493/22; C07F 7/1844

(57) ABSTRACT

Novel processes are disclosed for the preparation of eribulin mesylate. Novel intermediate compounds used in the processes for making eribulin mesylate as well as processes for making the intermediates are disclosed.

### CROSS-REFERENCE TO RELATED APPLICATIONS

This application claims the benefit of U.S. Provisional Patent Application Ser. No. 62/358,921 filed Jul. 6, 2016, the entirety of which is incorporated herein by reference.

### FIELD OF THE INVENTION

This invention relates to methods of making eribulin mesylate and intermediates thereof.

### BACKGROUND OF THE INVENTION

Eribulin mesylate is a chemotherapeutic compound which inhibits the growth phase of microtubules leading to G₂/M cell-cycle block, disruption of mitotic spindles and apoptotic cell death after prolonged mitotic blockage.

### SUMMARY OF THE INVENTION

Embodiments of the present disclosure relate to novel processes for making eribulin mesylate and intermediates thereof.

In accordance with various embodiments, novel processes are disclosed for the preparation of eribulin mesylate of Formula I

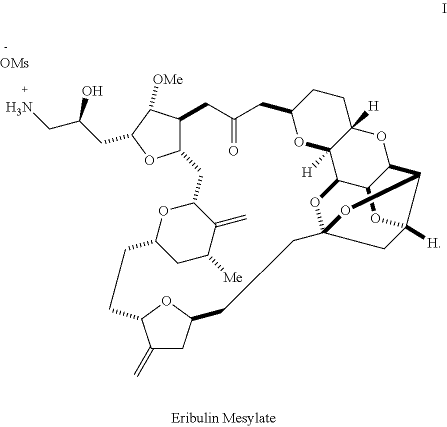

In accordance with further embodiments, a compound of formula F-5, or a salt thereof, and processes for making same are disclosed.

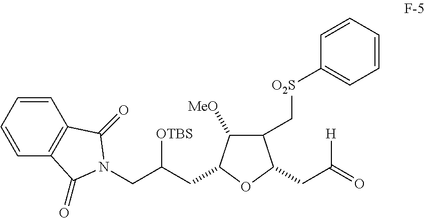

In accordance with further embodiments, a compound of formula 6a, or a salt thereof, and processes for making same are disclosed.

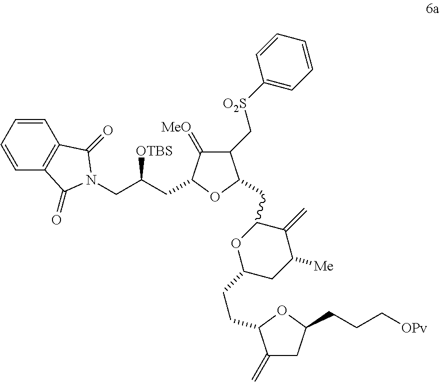

In still further embodiments, a compound of the formula 8n, or a salt thereof, and processes for making same are disclosed.

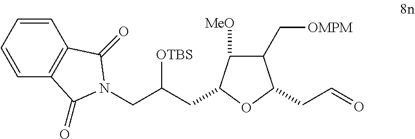

### DETAILED DESCRIPTION OF THE INVENTION

The present invention now will be described more fully hereinafter with reference to the accompanying examples and experiments, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The description of the molecules disclosed herein has been made using abbreviations that should be known to a skilled worker or can be determined. Some of the abbreviations used include: Ph for phenyl (C₆H₅—), Ar for aryl, which has been described herein, Ac for acetyl (CH₃C(═O)—), t-Bu for tert-butyl ((CH₃)₃C—), Et₃N for triethylamine ((CH₃CH₂)₃N), CDI for 1,1′-carbonyldiimidazole, PPh₃ for triphenylphosphine ((C₆H₅)₃P), Et for ethyl (C₂H₅—), SO2Ph for —SO₂C₆H₅, Me for methyl (CH₃—), MeO for methoxy (CH₃O), MeOH for methanol (CH₃OH), TBSO=OTBS=TBDMSO=OTBDMS for tert-butyldimethyl siloxy (((CH₃)₃C)(CH₃)₂SiO)—, Boc₂O for tert-butyl pyrocarbonate, NaIO₄ for sodium periodate, TMSN₃ for trimethylsilyl azide, Bn for benzyl (C₆H₅CH₂—), TMSI for trimethylsilyliodide ((CH₃)₃SiI), KHMDS for potassium hexamethyldisilazide, TBAF for tetra-butyl ammonium fluoride, mCPBA for meta-chloroperoxybenzoic acid, DMAP for dimethylaminopyridine, TsCI for tosyl chloride, and DMF for dimethylformamide.

In one embodiment, a process is disclosed for preparation of the compound of formula F-5, or a salt thereof.

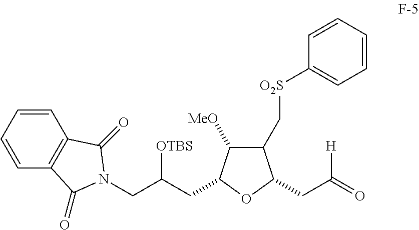

An exemplary synthetic route for making a compound of formula F-5 is outlined in Scheme 1:

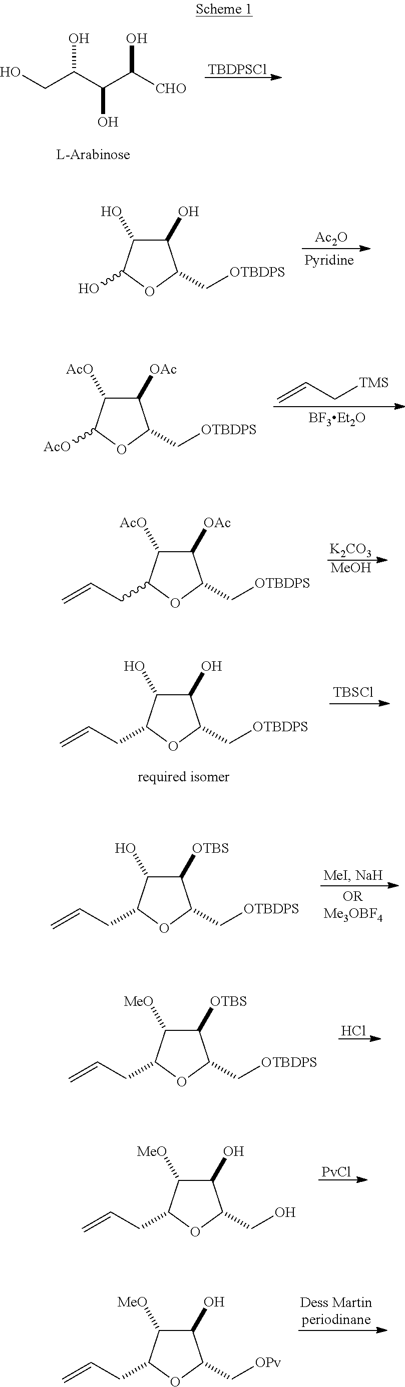

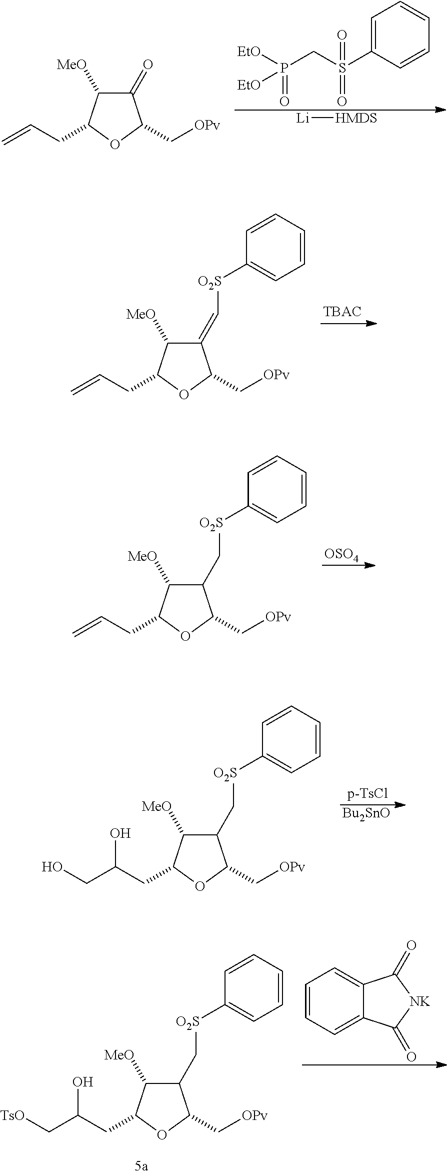

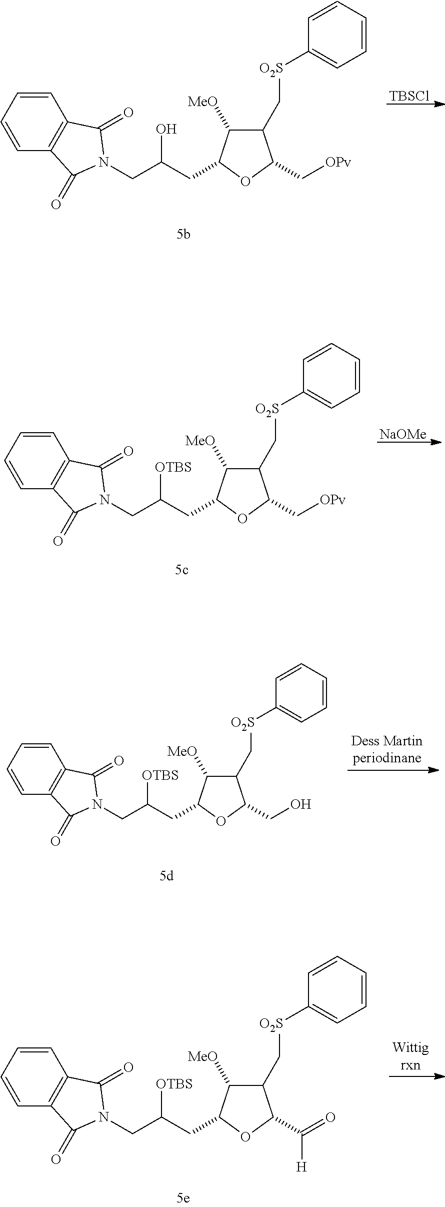

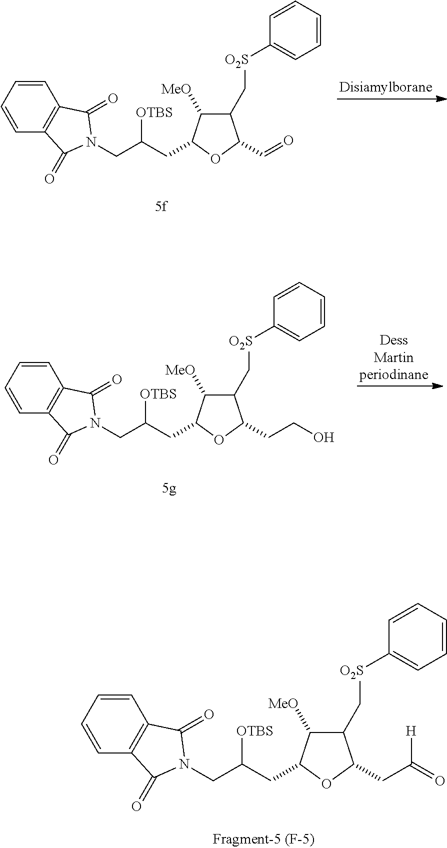 [SMILES: CO[C@@H]1C(CS(=O)(=O)c2ccccc2)[C@H](C=O)O[C@@H]1CC(CN1C(=O)c2ccccc2C1=O)O[Si](C)(C)C(C)(C)C ; CO[C@@H]1C(CS(=O)(=O)c2ccccc2)[C@H](CCO)O[C@@H]1CC(CN1C(=O)c2ccccc2C1=O)O[Si](C)(C)C(C)(C)C ; CO[C@@H]1C(CS(=O)(=O)c2ccccc2)[C@H](CC=O)O[C@@H]1CC(CN1C(=O)c2ccccc2C1=O)O[Si](C)(C)C(C)(C)C]

A process of converting the terminal alcohol of the compound of formula 4a into an amine, substituted amine or phthalimide group to form the compound of formula F-5 is not particularly limited.

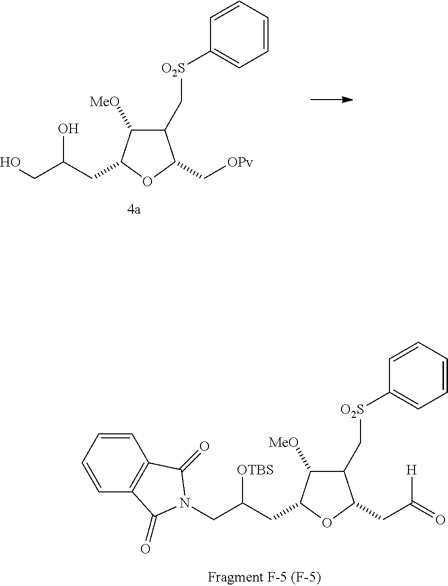

In one embodiment, for example and without limitation, the conversion is carried out by converting the alcohol into a leaving group, as described herein, to form an intermediate 5a, followed by substitution of the leaving group by an amine or phthalimide group or other nitrogen based nucleophile to form the compound of formula 5b.

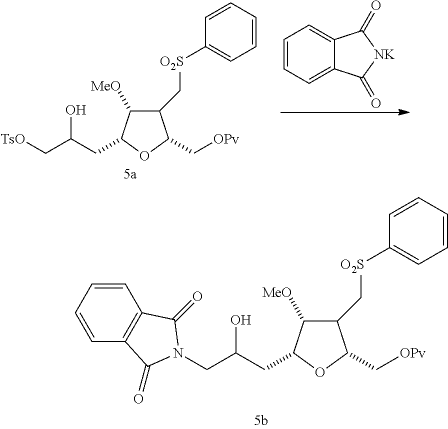

The amine protecting group as used herein is not particularly limited and should be known to a person of skill in the art. In one embodiment, for example and without limitation, an amine protecting group can include toluenesulfonyl chloride (p-TsCl), carbobenzyloxy (Cbz), p-methoxybenzyloxy carbonyl (Moz), tert-butoxycarbonyl (t-BOC), 9-fluorenylmethoxycarbonyl (FMOC), acetyl (Ac), benzoyl (Bz), carbamate, p-methoxybenzyl (PMB), 3,4-dimethoxybenzyl (DMPM) or p-methoxyphenyl (PMP). In a further embodiment, the amine protecting group is toluenesulfonyl chloride (p-TsCl).

The process for protecting of the alcohol group is not particularly limited to by TBSCl. In one embodiment, for example and without limitation, the conversion is carried out by converting the alcohol into a leaving group, as described herein, to form an intermediate 5c, followed by de-protection of the alcohol group. De-protection methods include treatment of the pivaloyl chloride (PvCl) group with sodium methoxide to form the compound of formula 5d.

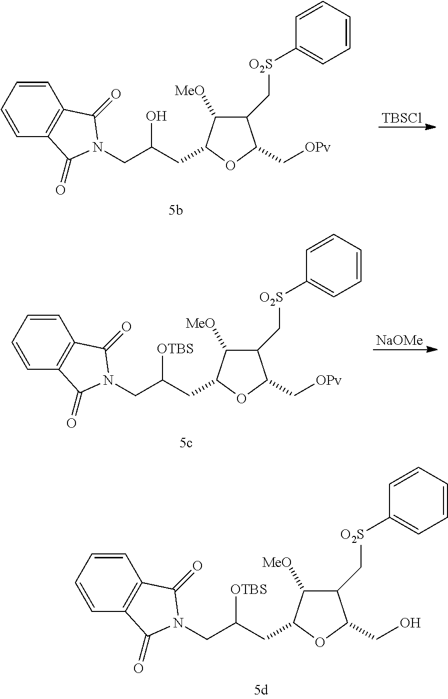

In one embodiment, for example and without limitation, the alcohol is oxidized to a ketone prior to conversion to the compound of formula 5d. The oxidation of the alcohol is not particularly limited, and should be known to a skilled worker or can be determined without undue experimentation. In one embodiment, for example and without limitation, the oxidation is performed using a chromium-based reagent, such as but not limited to Collins reagent, pyridinium dichromate (PDC) or pyridinium chlorochromate (PCC); activated dimethyl sulfoxide (DMSO), such as by Swern oxidation, Moffatt oxidation or Doering oxidation; or hypervalent iodine compounds, such as Dess-Martin periodinane, 2- iodoxybenzoic acid, etc.

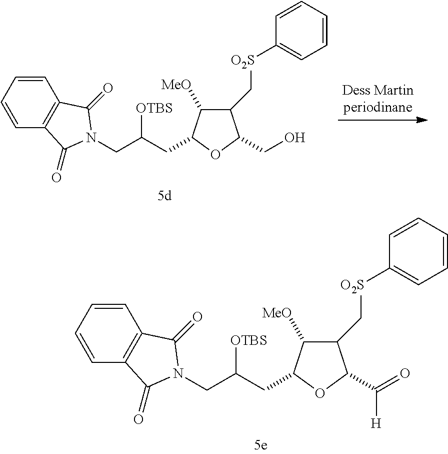 [SMILES: CO[C@@H]1C(CS(=O)(=O)c2ccccc2)[C@H](CO)O[C@@H]1CC(CN1C(=O)c2ccccc2C1=O)O[Si](C)(C)C(C)(C)C ; CO[C@@H]1C(CS(=O)(=O)c2ccccc2)[C@H](C=O)O[C@@H]1CC(CN1C(=O)c2ccccc2C1=O)O[Si](C)(C)C(C)(C)C]

Following oxidation of the alcohol to a ketone, the ketone functional group can be, in one embodiment, for example and without limitation, converted into an alkene. The reaction to convert a ketone to an alkene is not particularly limited, and should be known to a skilled worker or can be determined without undue experimentation. In one embodiment, for example and without limitation, the ketone can be converted into an alkene using the Peterson olefination, the Wittig reaction or the like. In a further embodiment, for example and without limitation, the ketone is converted into an alkene using CH₃PPh₃Br.

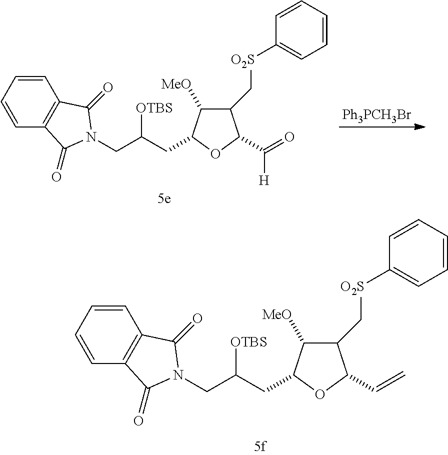

Upon formation of the alkene, the compound can be reduced to alkane using a hydroboration-oxidation reaction. The hydroboration-oxidation reaction has been developed for converting the alkene into an alcohol by the net addition of water across the double bond. In one embodiment, for example and without limitation, the hydroboration-oxidation reaction is carried out using a disiamylborane. The hydroboration-oxidation reaction source used is not particularly limited and should be known to a skilled worker or can be determined without undue experimentation. In one embodiment, for example and without limitation, the disiamylborane is employed.

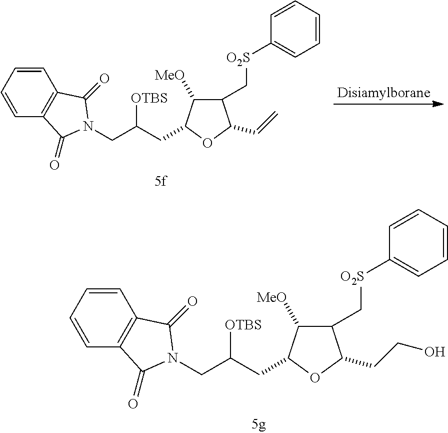

In one embodiment, for example and without limitation, the alcohol is oxidized to a ketone prior to conversion to the compound of formula 5g. The oxidation of the alcohol is not particularly limited, and should be known to a skilled worker or can be determined. In one embodiment, for example and without limitation, the oxidation is performed using a chromium-based reagent, such as Collins reagent, pyridinium dichromate (PDC) or pyridinium chlorochromate (PCC); activated dimethyl sulfoxide (DMSO), such as by Swern oxidation, Moffatt oxidation or Doering oxidation; or hypervalent iodine compounds, such as but not limited to Dess-Martin periodinane or 2- iodoxybenzoic acid.

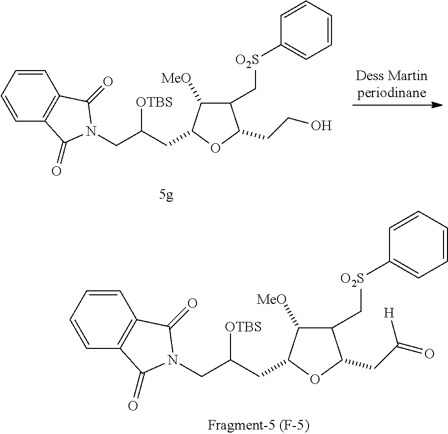

Various methods are provided herein for the synthesis of eribulin mesylate. Schemes 2, 3 and 5 outline exemplary synthetic routes.

For example, as outlined in Scheme 2, de-protection of the silyl ether hydroxyl protecting groups (e.g., TBS) of a compound of formula 6e followed by equilibration furnishes a compound of formula 6f. Ketalization of a compound of formula 6g provides a compound of formula 6h. Activation of the primary alcohol (e.g., as the tosylate) resulting in a compound of formula 6i, followed by reduction of the keto functionality, provides a compound of formula 6j. Further reduction of the phthalimide group of the compound of formula 6j to an amine group results in a compound of formula 6k, and reduction in the presence of hydrazine followed by de-protection provides eribulin.

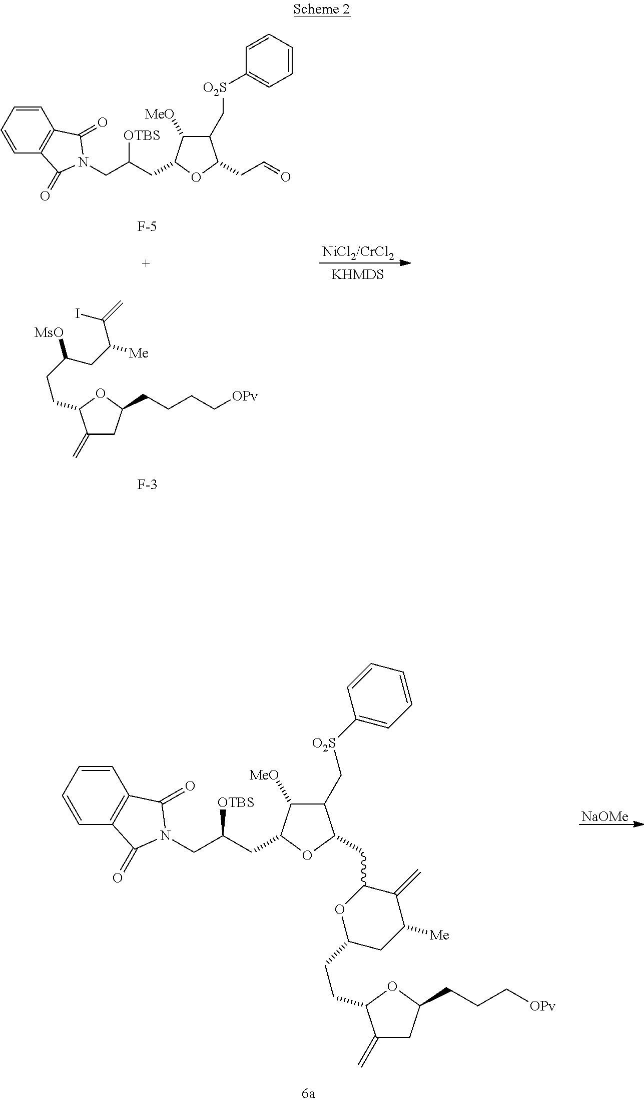

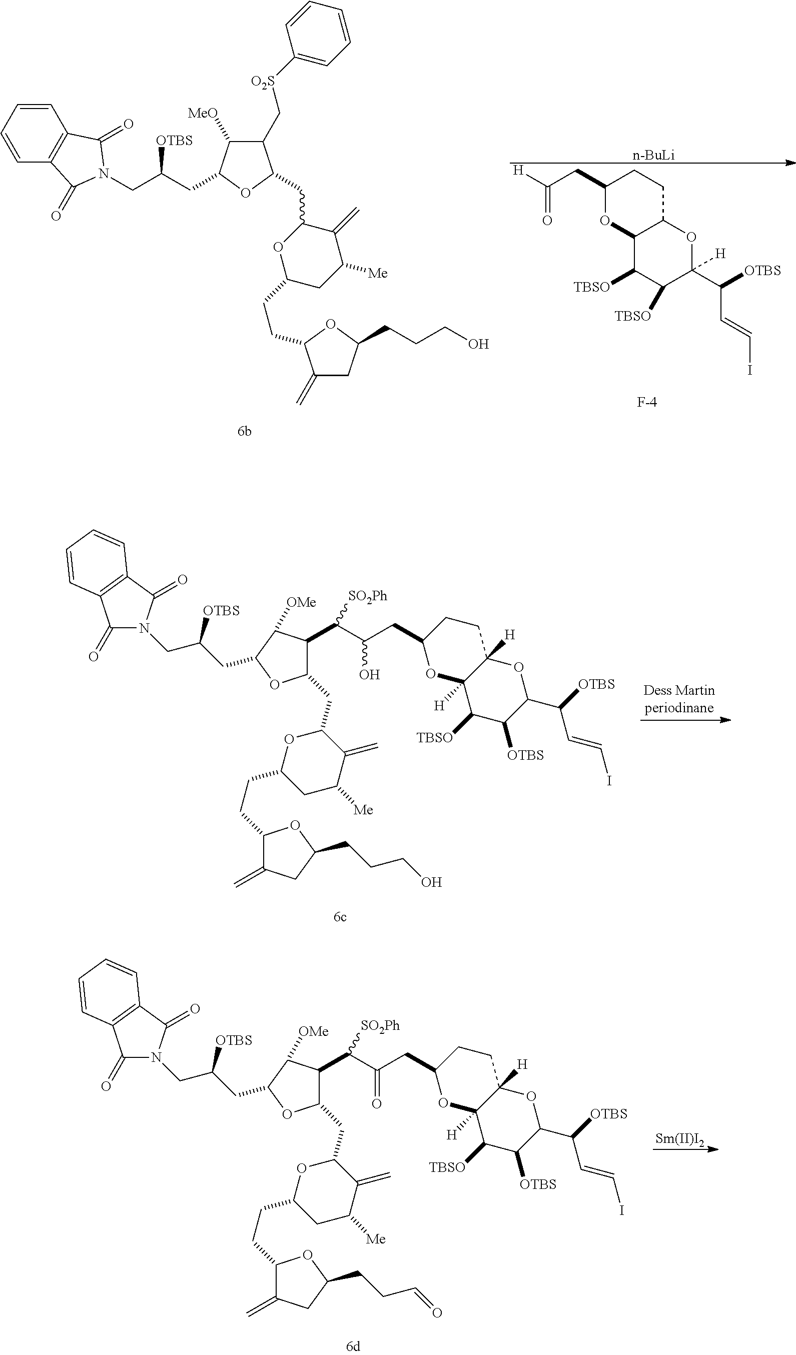

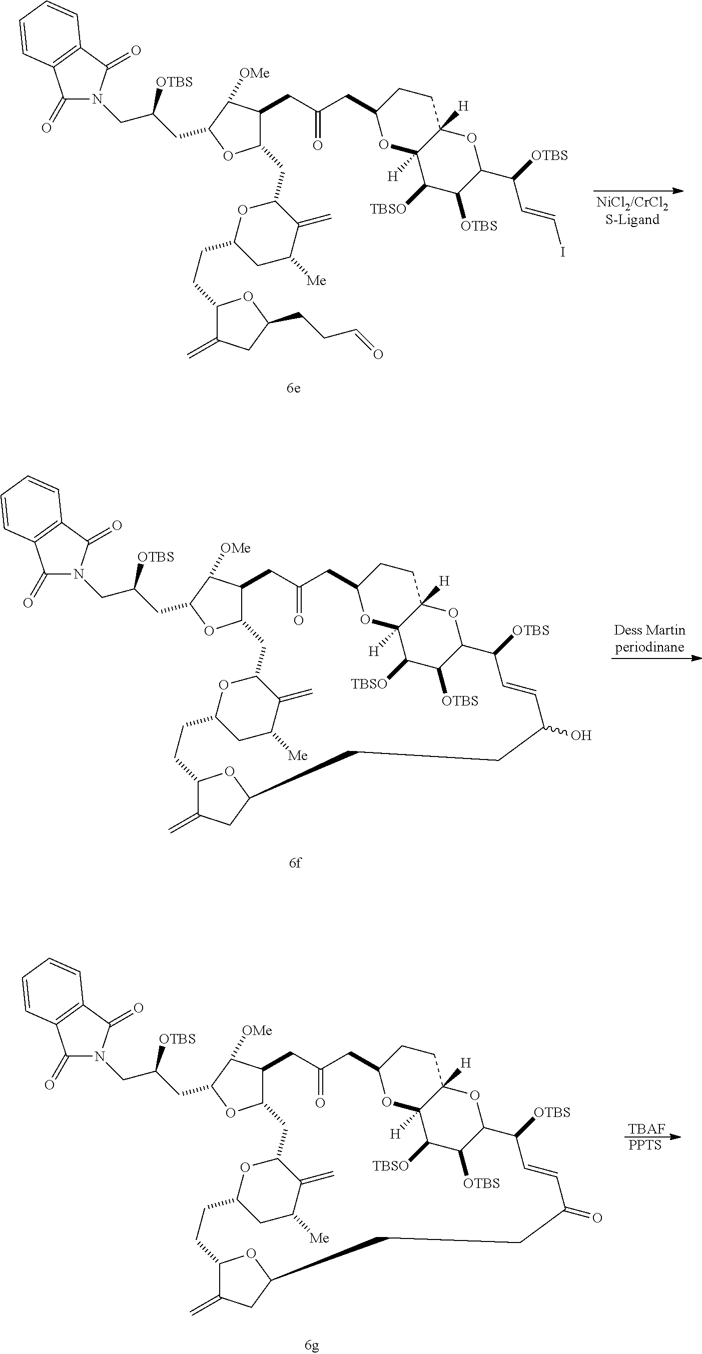

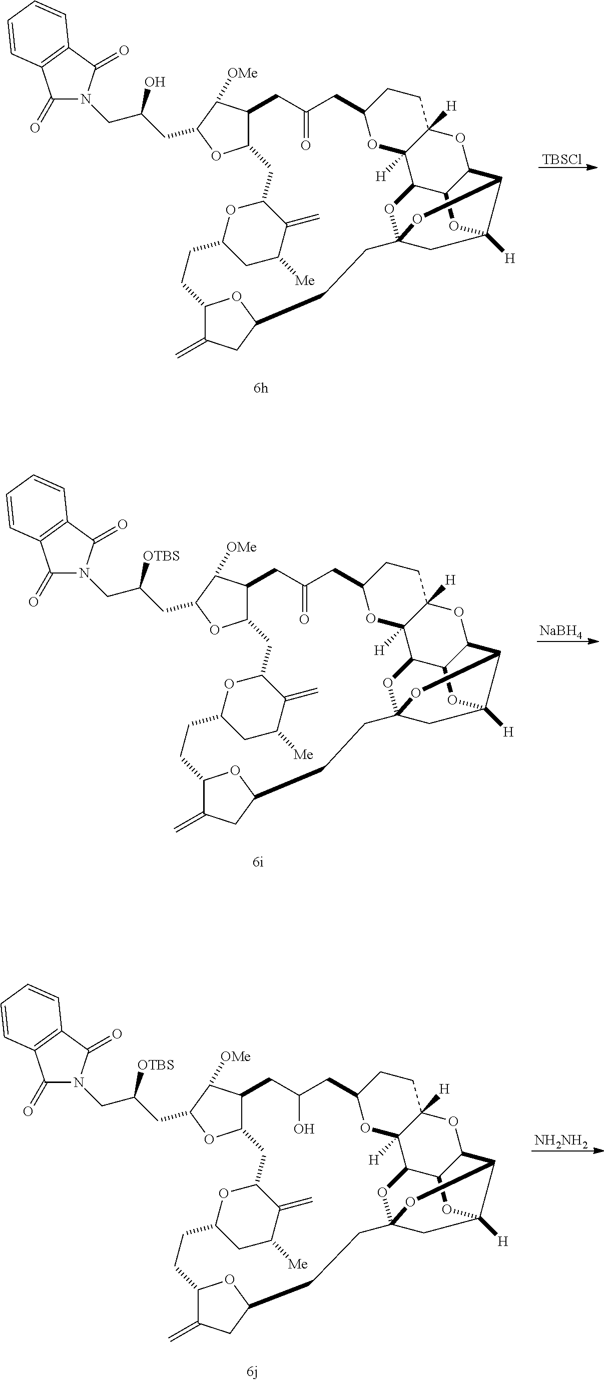

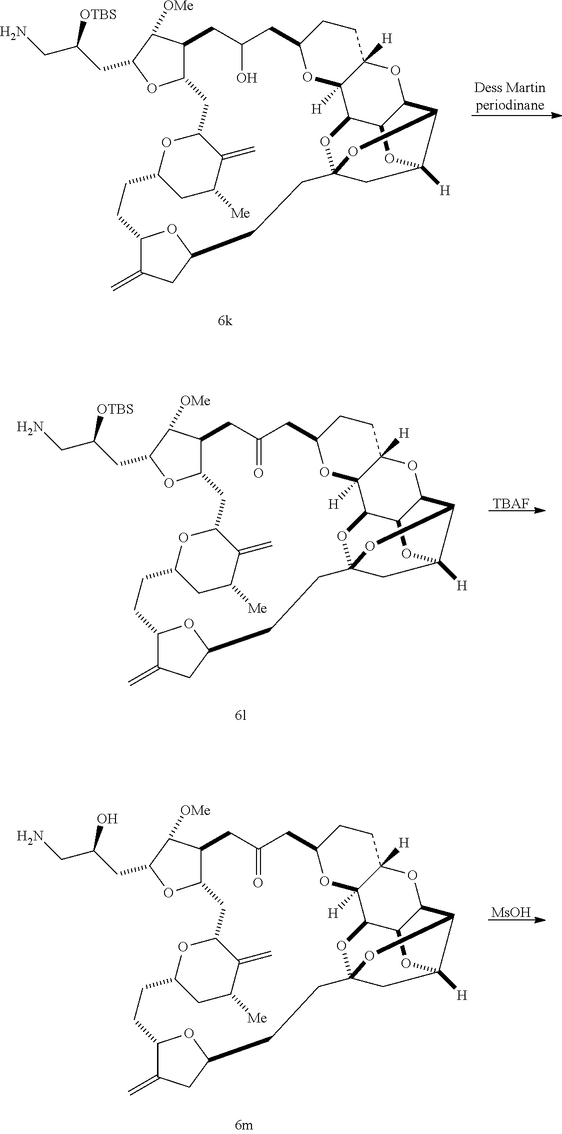

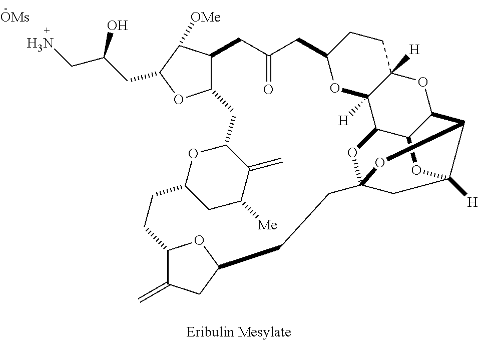

In some embodiments processes are disclosed for preparation of a compound of formula 6a, or a pharmaceutically acceptable salt thereof. The process involves coupling a compound of formula F-5 with a compound of formula F3, to form the compound of formula 6a.

The method of coupling the compound of formula F-5 with a compound of formula F3 is not particularly limited. In one embodiment, for example and without limitation, the coupling reaction is performed using a nickel/chromium catalyst, such as in the Nozaki-Hiyama-Kishi reaction. In a still further embodiment, for example and without limitation, the catalyst used for the coupling reaction is NiCI2/CrCI2. In another embodiment, for example and without limitation, the coupling reaction performed is a Grignard reaction.

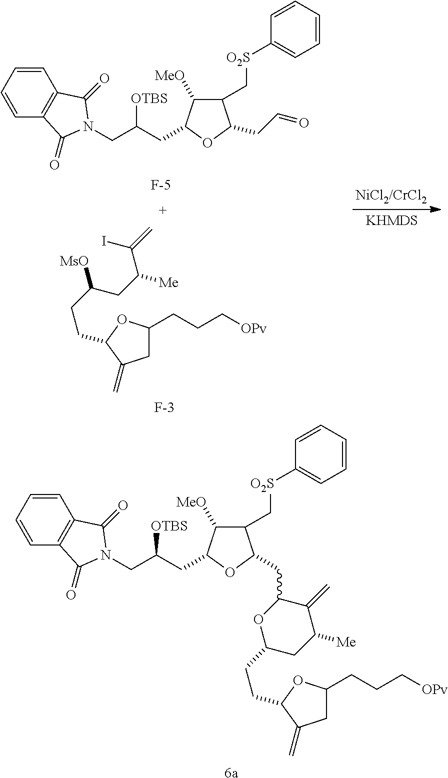

The alcohol group of the compound of formula 6a can be de-protected using conditions that should be known to a skilled worker. For the de-protection of the alcohol group, methods include treatment of the pivaloyl chloride (PvCl) group with sodium methoxide to form the compound of formula 6b. In the embodiment disclosed, the de-protection is performed by sodium methoxide, to obtain the compound of formula 6b. Coupling of the compound of formula 6b with the compound of formula F4 can be performed under basic conditions in the presence of n-butyllithium (n-BuLi) to form an intermediate alcohol 6c. The oxidation of the intermediate alcohol 6c is not particularly limited, for example and without limitation, the oxidation is performed using a chromium-based reagent, such as Collins reagent, pyridinium dichromate (PDC) or pyridinium chlorochromate (PCC); activated dimethyl sulfoxide (DMSO), such as by Swern oxidation, Moffatt oxidation or Doering oxidation; or hypervalent iodine compounds, such as but not limited to, Dess-Martin periodinane or 2- iodoxybenzoic acid to result in formation of the compound of formula 6d. The resulting ketone-aldehyde intermediate compound of formula 6d is followed by reduction of the arylsulfonyl moiety using a reducing agent, for example and without limitation SmI₂ to afford intermediate compound of formula 6e.

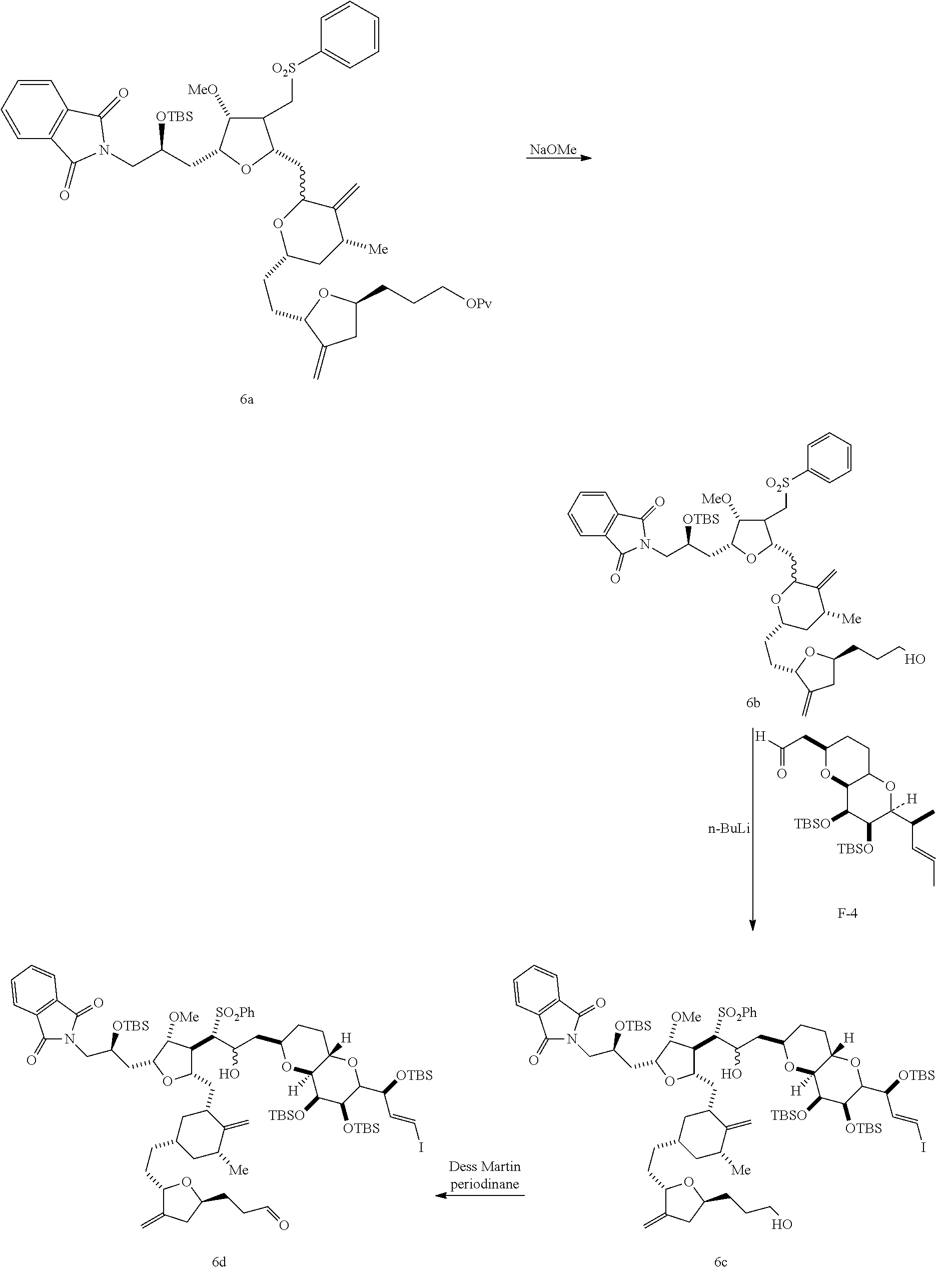

In one or more embodiments, the compound of formula 6e is subjected to an intramolecular coupling reaction, under conditions similar as performed using a Ni/Cr catalyst to obtain a compound of formula 6f In one embodiment, for example and without limitation, the coupling reaction is performed using a nickel/chromium catalyst, such as in the Nozaki-Hiyama-Kishi reaction. In a still further embodiment, for example and without limitation, the catalyst used for the coupling reaction is NiCl₂/CrCl₂. In another embodiment, for example and without limitation, the coupling reaction performed is a Grignard reaction.

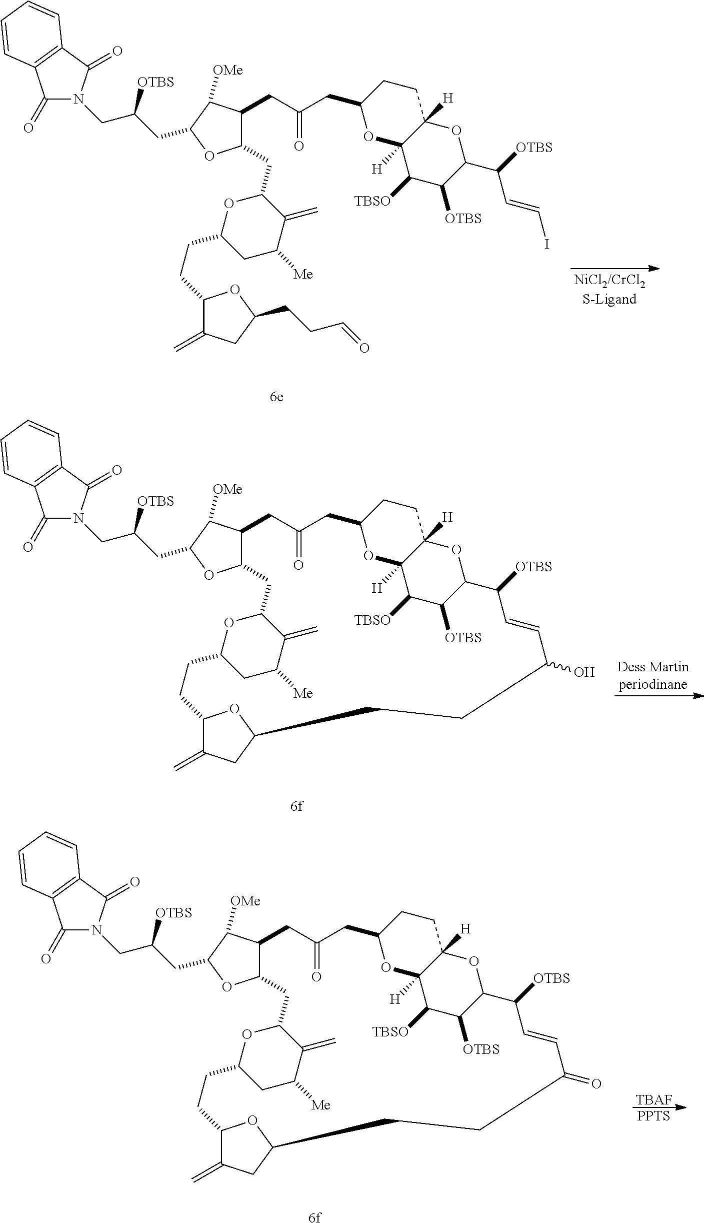

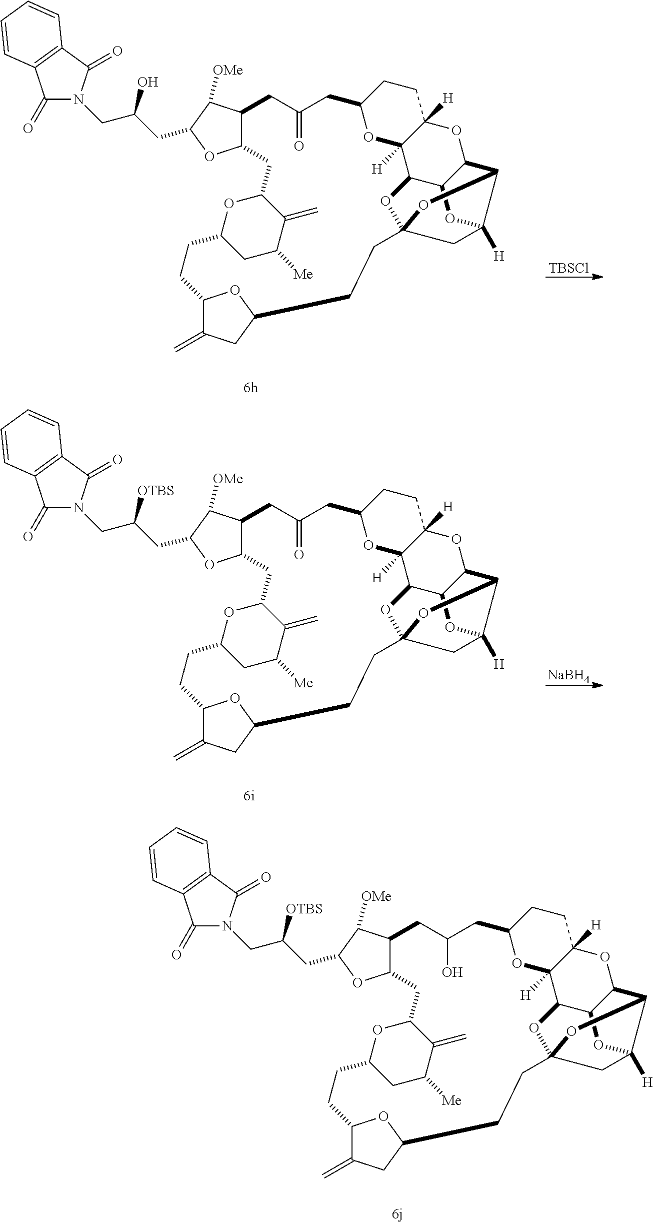

Desilylation of the compound of formula 6g can be performed using reagents using a fluoride source. In one embodiment, for example and without limitation, the desilylation is performed using tetra-butyl ammonium fluoride (TBAF). Intramolecular cyclization of the resultant alcohol intermediate can be performed under acidic conditions.

In one embodiment the polycyclic ring system was, cleanly and effectively, incorporated on treatment with TBAF then p-TsOH.Py (PPTS). The regioselectivity of the Michael reaction was exclusive for the desired five-membered ring-formation, whereas the stereoselectivity was approximately favoring the desired diastereomer. The undesired Michael adduct, separated from the desired product after PPTS treatment, could be recycled under TBAF conditions.

Treatment of a compound of formula 6g with a fluoride source (e.g., tetrabutylammonium fluoride) and equilibration with a conjugate acid of imidazole (e.g., imidazole hydrochloride), in tetrahydrofuran as solvent and further intramolecular cyclization (e.g., in dichloromethane) with a conjugate acid of pyridine (e.g., pyridinium p-toluenesulfonate (PPTS)), followed by crystallization from acetonitrile and water, provides compound of formula 6h. The method of performing the intramolecular cyclization reaction as disclosed herein is not particularly limited. In one embodiment, for example and without limitation, the intramolecular cyclization reaction is performed using an acid. The type of acid used is also not particularly limited. In one embodiment, for example and without limitation, the acid is a mild acid that is also non-nucleophilic, and can be, for example but not limited to, pyridinium p-toluenesulfonate (PPTS), trialkyl ammonium sulfate and weak carboxylic acids, such as, for example and without limitation, acetic acid. Following the cyclization reaction, the reaction product can be treated with a base to neutralize the reaction mixture. The base used is not particularly limited. In one embodiment, the base is, for example, cesium carbonate (Cs₂CO₃). In addition, alkali metal based bases, such as an alkali metal carbonates, phosphates etc. can also be used.

Activation of a compound of formula 6h with tert-butyldimethylchlorosilane and imidazole or pyridine by reacting compound of formula 6h with tert-butyldimethylchlorosilane and a base (e.g., imidazole, pyridine) provides a compound of formula 6i.

The amination of a compound of formula 6j can be achieved through treatment with hydrazine, followed by reduction of the resulting phthalimide group under reaction conditions to produce a compound of formula 6k. Further the alcohol is oxidized to a ketone by Dess-Martin periodinane to produce a compound of formula 6l. Desilylation of the compound of formula 6l is performed using a fluoride source on treatment with TBAF to produce compound of formula 6m (Eribulin).

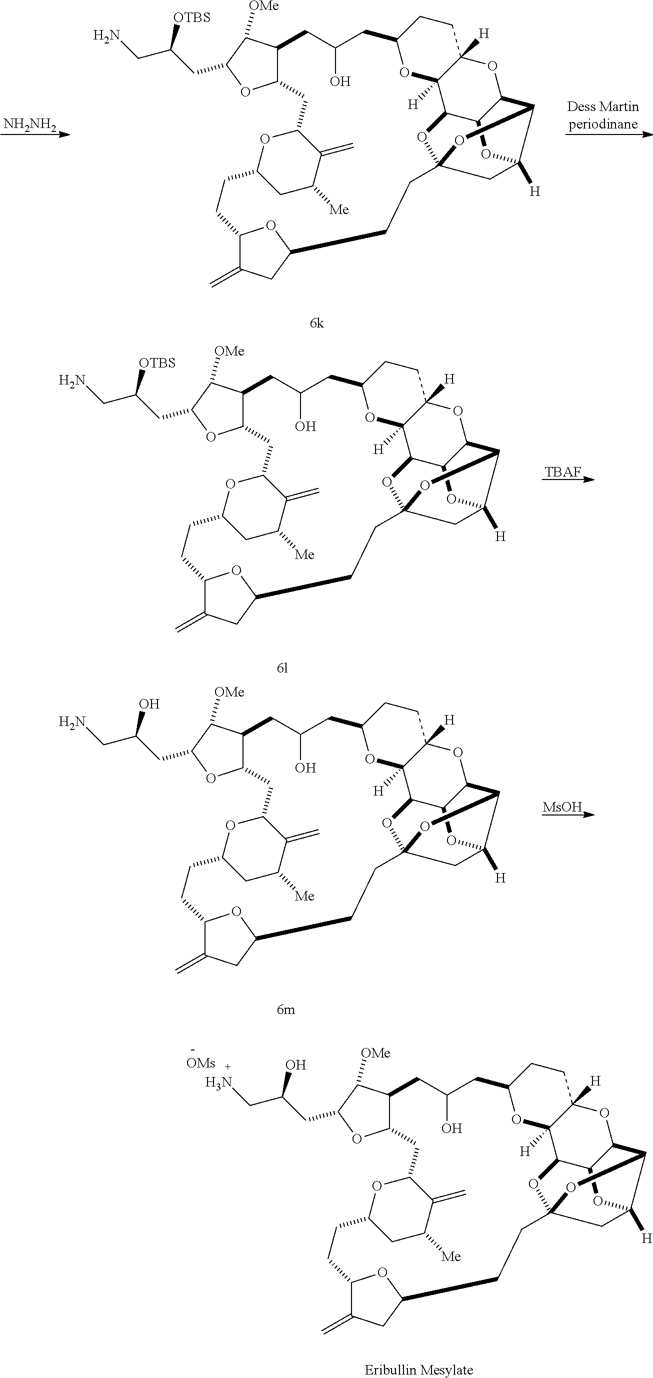

Pharmaceutically acceptable salts of eribulin (e.g., eribulin mesylate) can be formed by methods known in the art (e.g., in situ during the final isolation and purification of the compound or separately by reacting the free base group with a suitable acid). In one example, eribulin is treated with a solution of methanesulfonic acid (i.e., MsOH) and ammonium hydroxide in water and acetonitrile. The mixture is concentrated. The residue is dissolved in dichloromethane-pentane, and the solution is added to anhydrous pentane. The resulting precipitate is filtered and dried under high vacuum to provide eribulin mesylate.

In accordance with further exemplary embodiments, Schemes 3-5 are disclosed. Scheme 3 uses the same starting materials (compounds F-5 and F-3) as Scheme 2. Scheme 4 discloses a synthetic route for making the starting material of the Scheme 5 synthetic route for making eribulin mesylate.

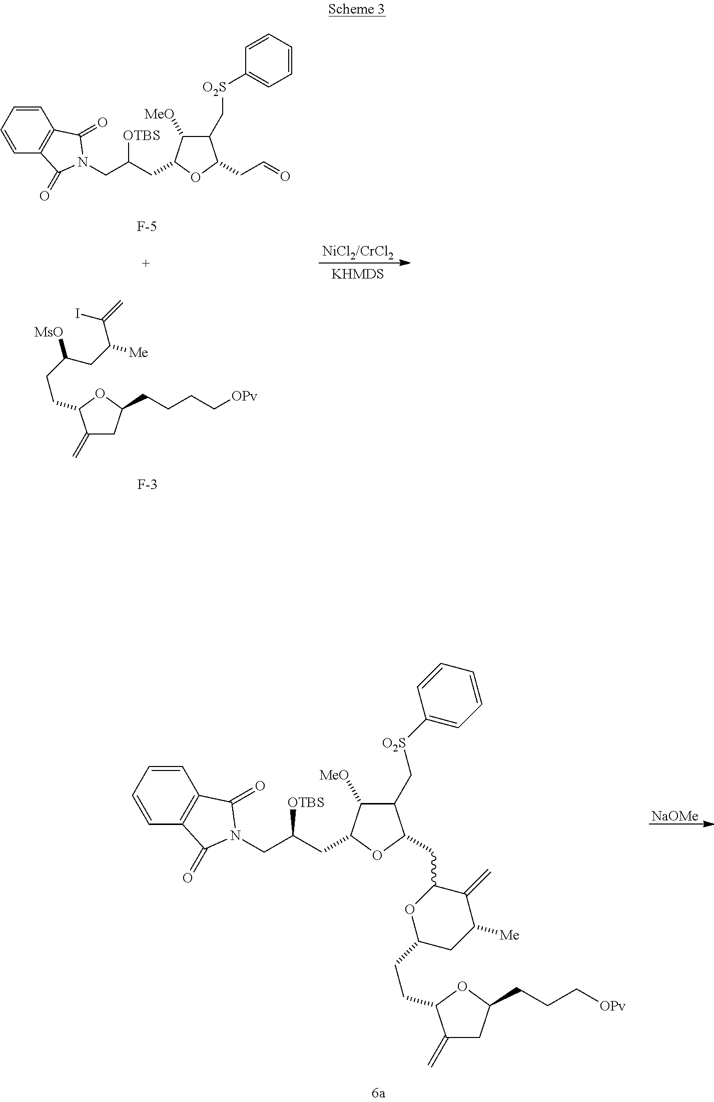

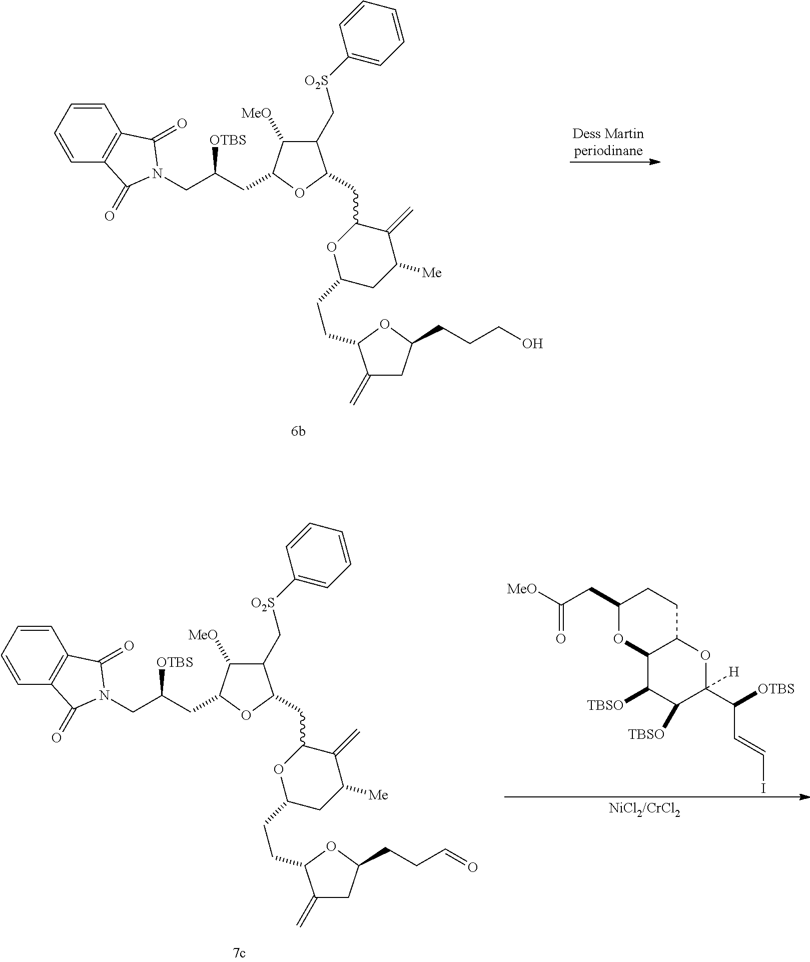

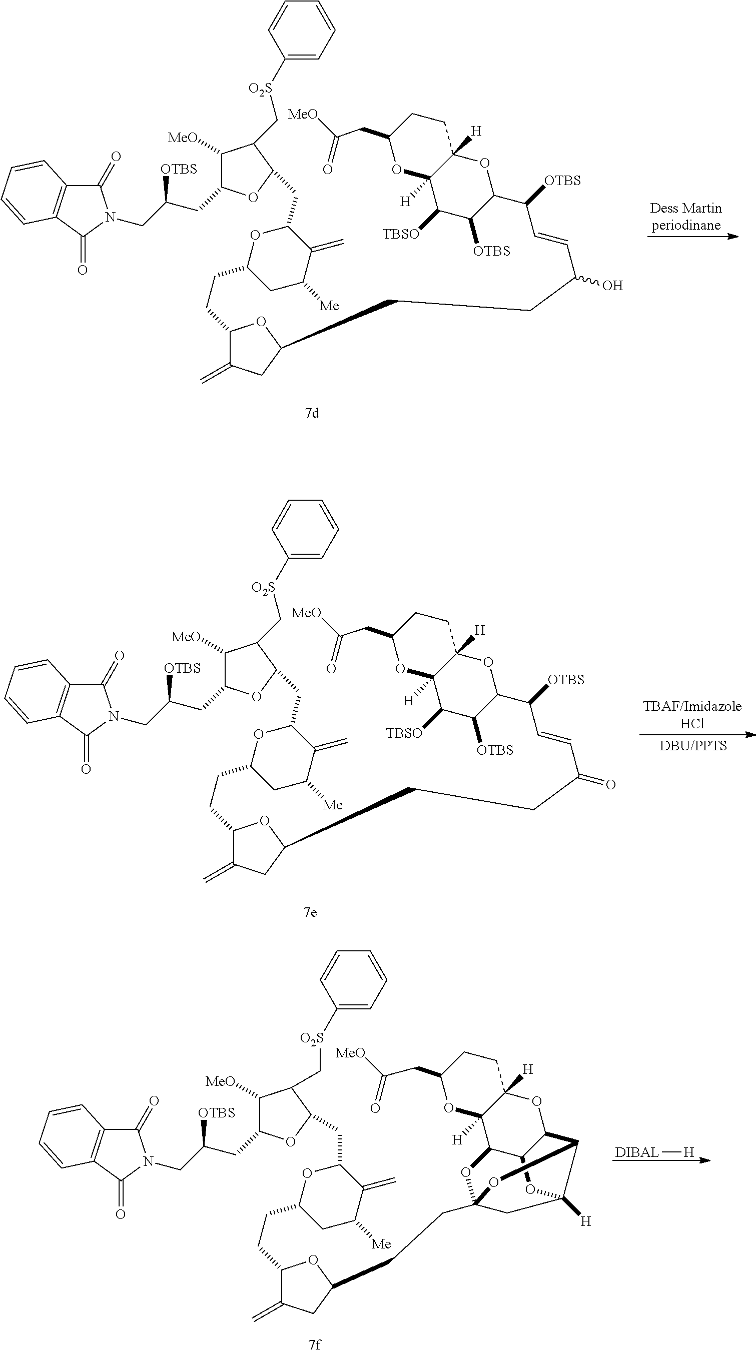

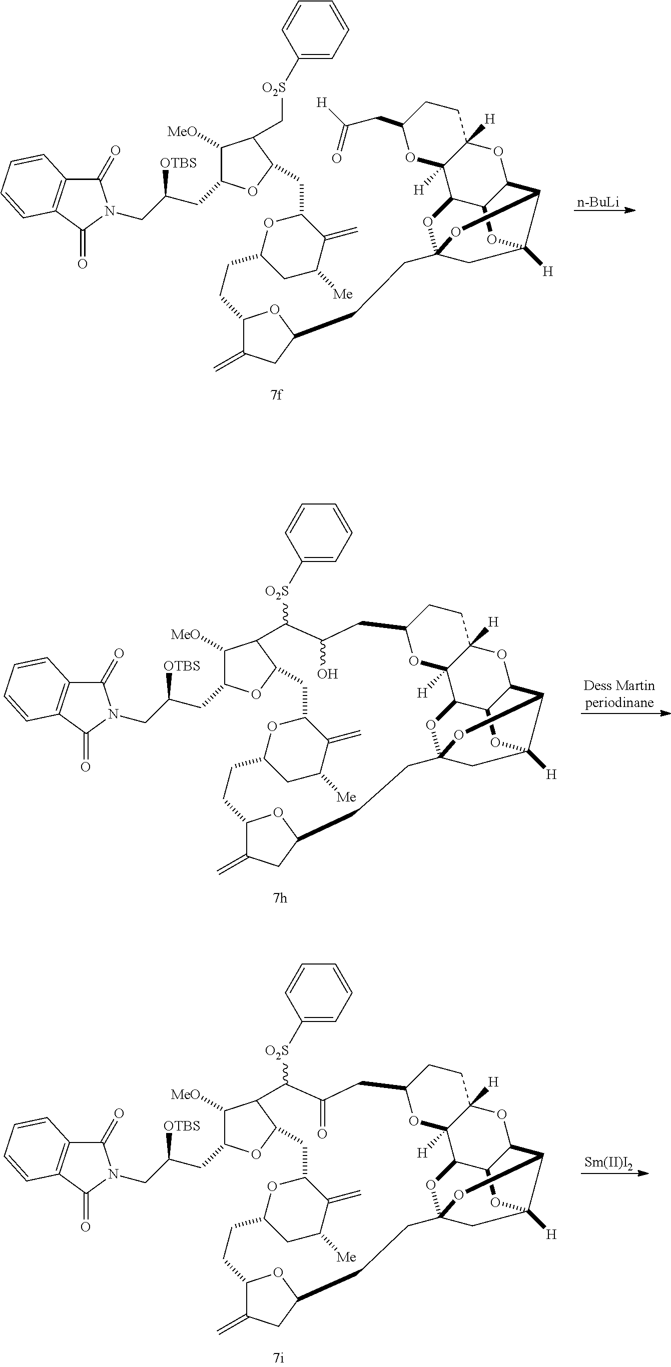

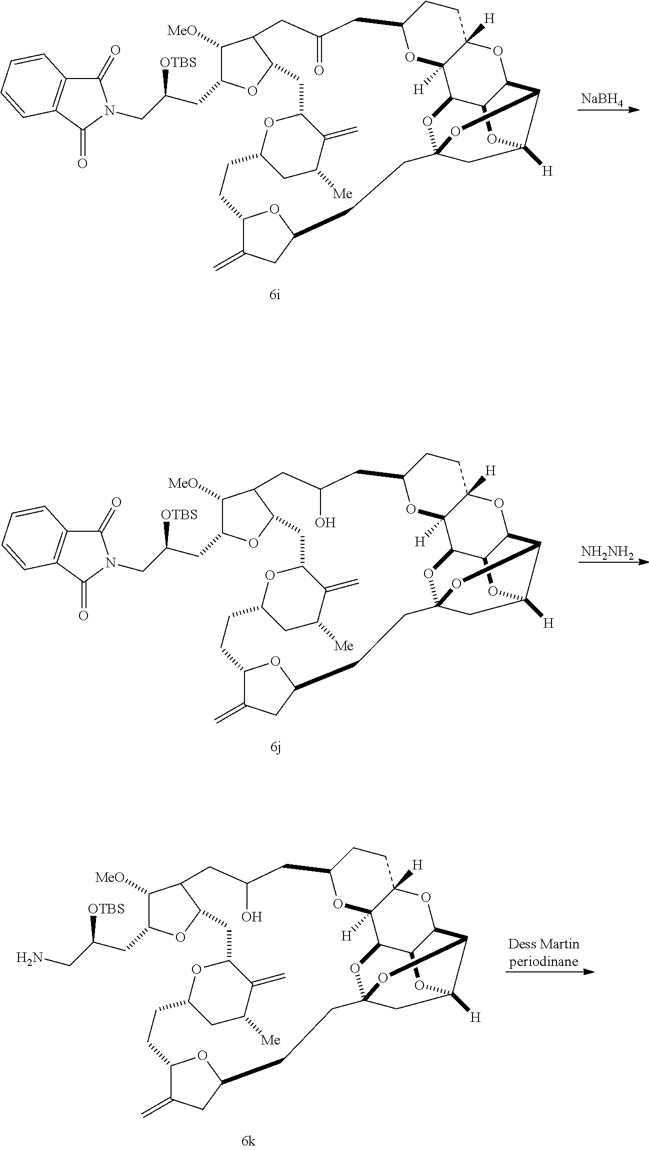

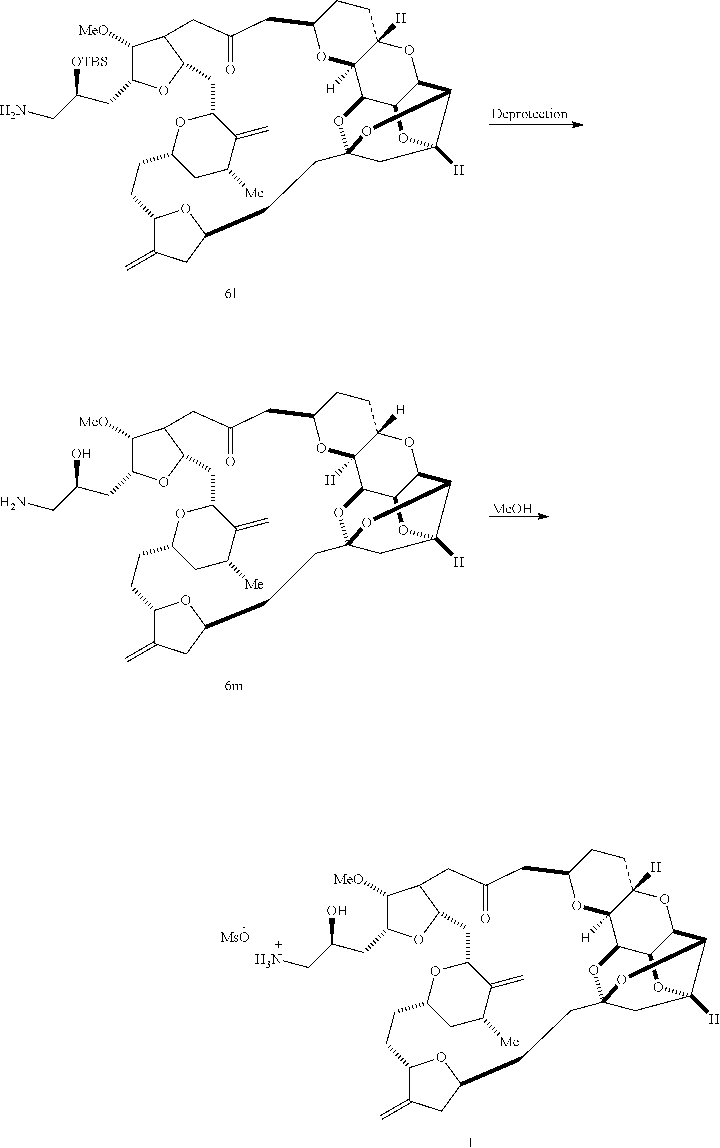

In one or more embodiments a method of making eribulin mesylate according to Scheme 3 includes the steps of:
a. reacting a compound of the formula F5

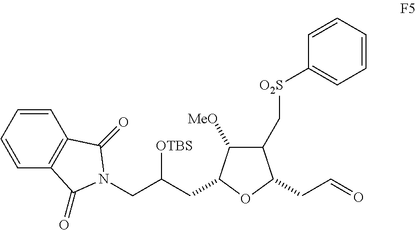

with a compound of the formula F3

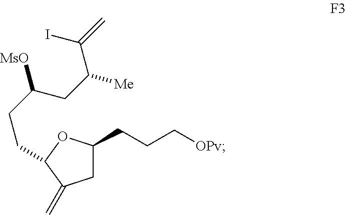

b. reacting the compound obtained in step a with sodium methoxide to produce a compound of the formula 6b

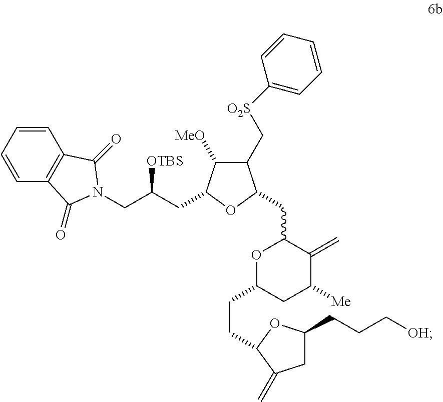

c. oxidizing the compound of formula 6b to produce a compound of the formula 7c

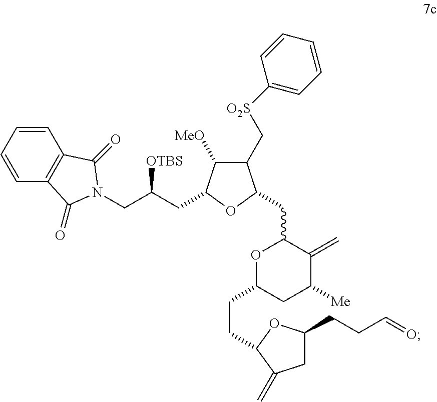

d. reacting a compound of the formula 7c with a compound of the formula F4

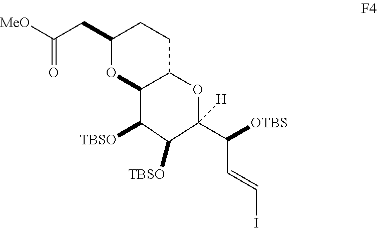

to produce a compound of the formula 7d

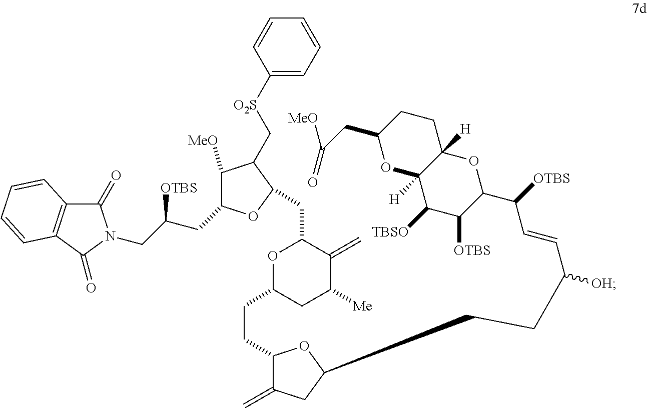

e. oxidizing the compound of formula 7d to produce a compound of the formula 7e

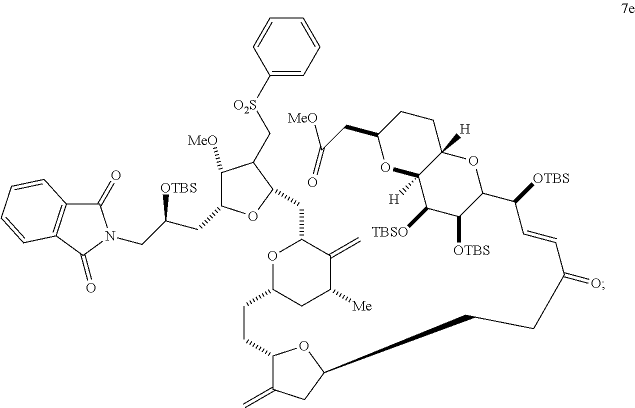

f desilylating and subjecting to an intramolecular cyclization reaction the compound of the formula 7e to produce a compound of the formula 7f

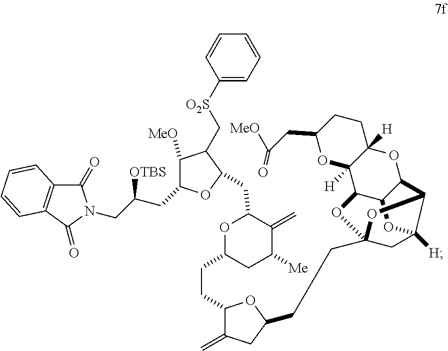

g. subjecting the compound of the formula 7f to a reducing agent to produce a compound of the formula 7g

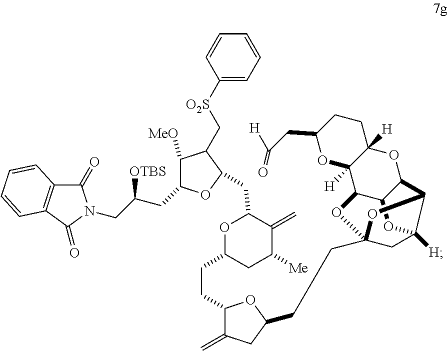

h. conducting an intramolecular coupling reaction of the compound of formula 7g to produce a compound of the formula 7h

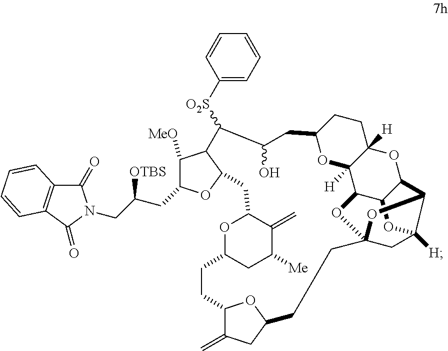

i. oxidizing the compound of the formula 7h to produce a compound of the formula 7i;

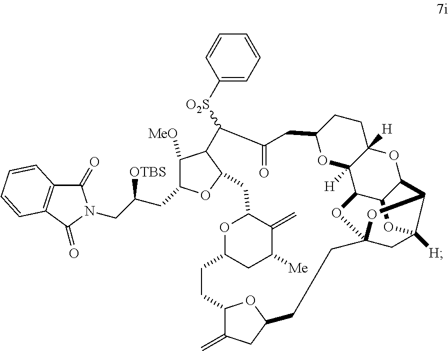

j. reducing the arylsulfonyl moiety of the compound of the formula 7i to obtain a compound of the formula 6i

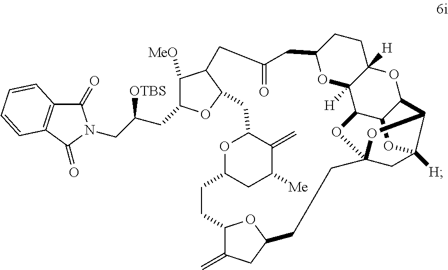

k. subjecting the compound of the formula 6i to a reducing agent to obtain a compound of the formula 6j;

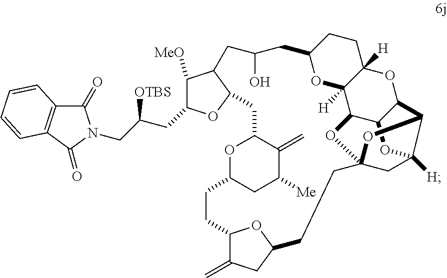

l. treating the compound of the formula 6j with a reducing agent operable to reduce the phthalimide group of the compound of formula 6j to obtain a compound of the formula 6k

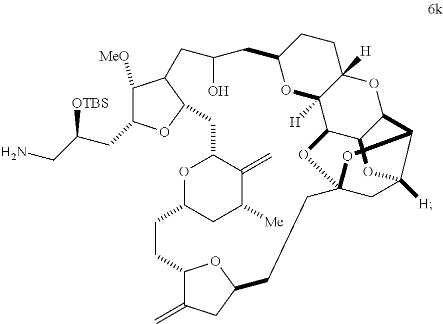

m. oxidizing the compound of the formula 6k to obtain a compound of the formula 6l

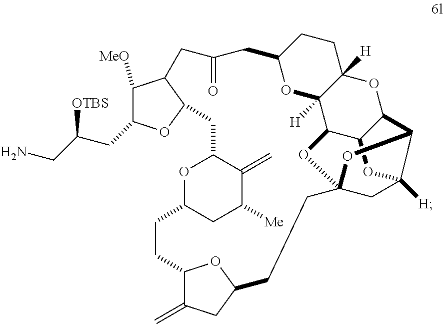

n. deprotecting the compound of the formula 6l to obtain a compound of the formula 6m

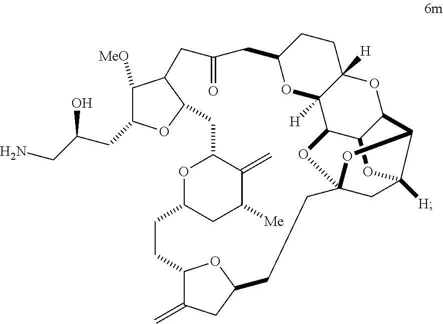

and
o. treating the compound of the formula 6m with methanesulfonic acid to obtain eribulin mesylate.

In accordance with further embodiments, a method is disclosed in Scheme 4 for preparing an intermediate compound of the formula 8n

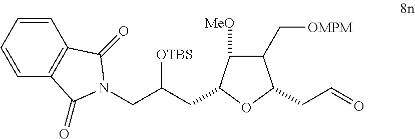

which may be employed as a starting material for the synthetic route shown in Scheme 5.

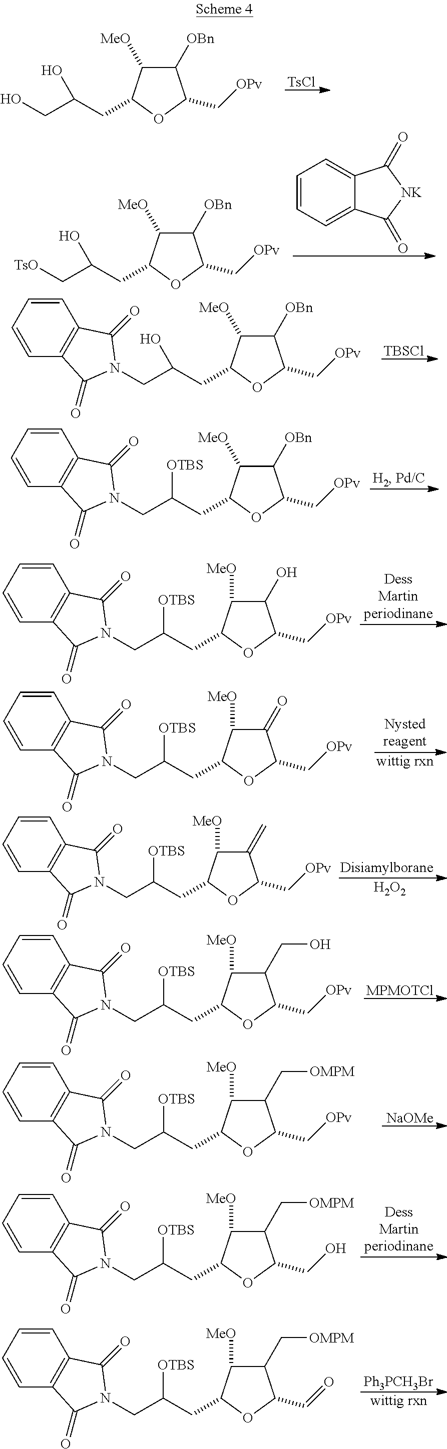

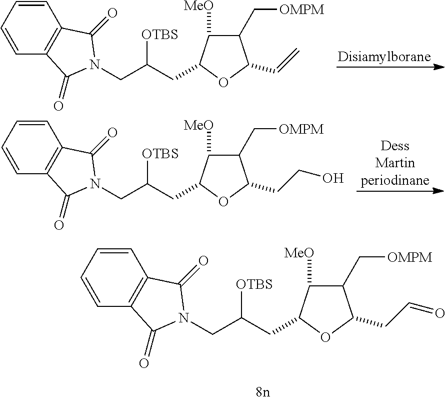

In one or more embodiments, a synthetic route according to Scheme 5, using the compound of the formula 8n obtained from Scheme 4, may be employed to obtain eribulin mesylate.

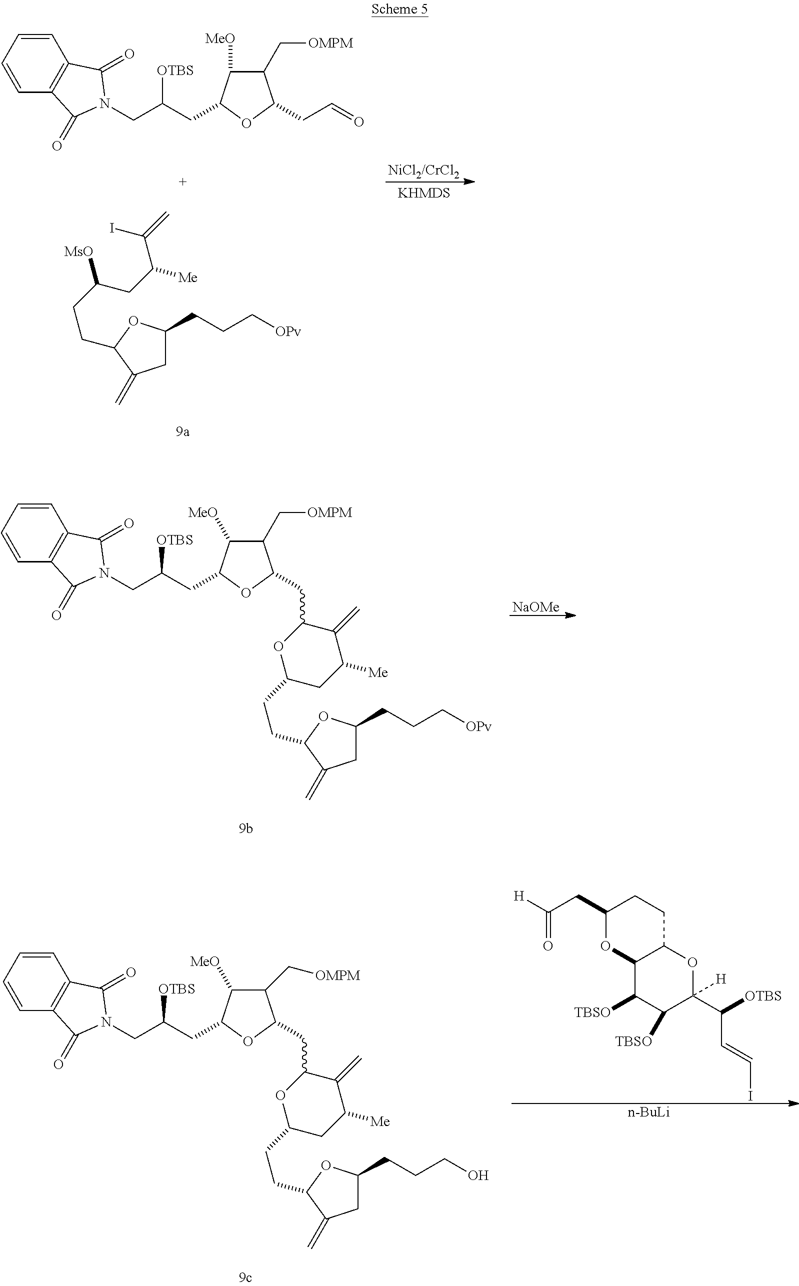

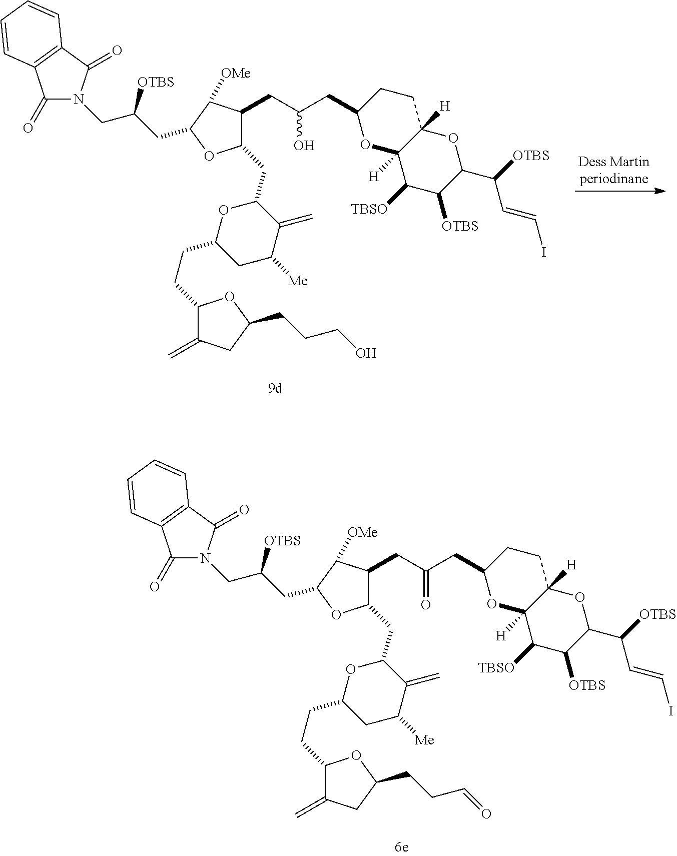

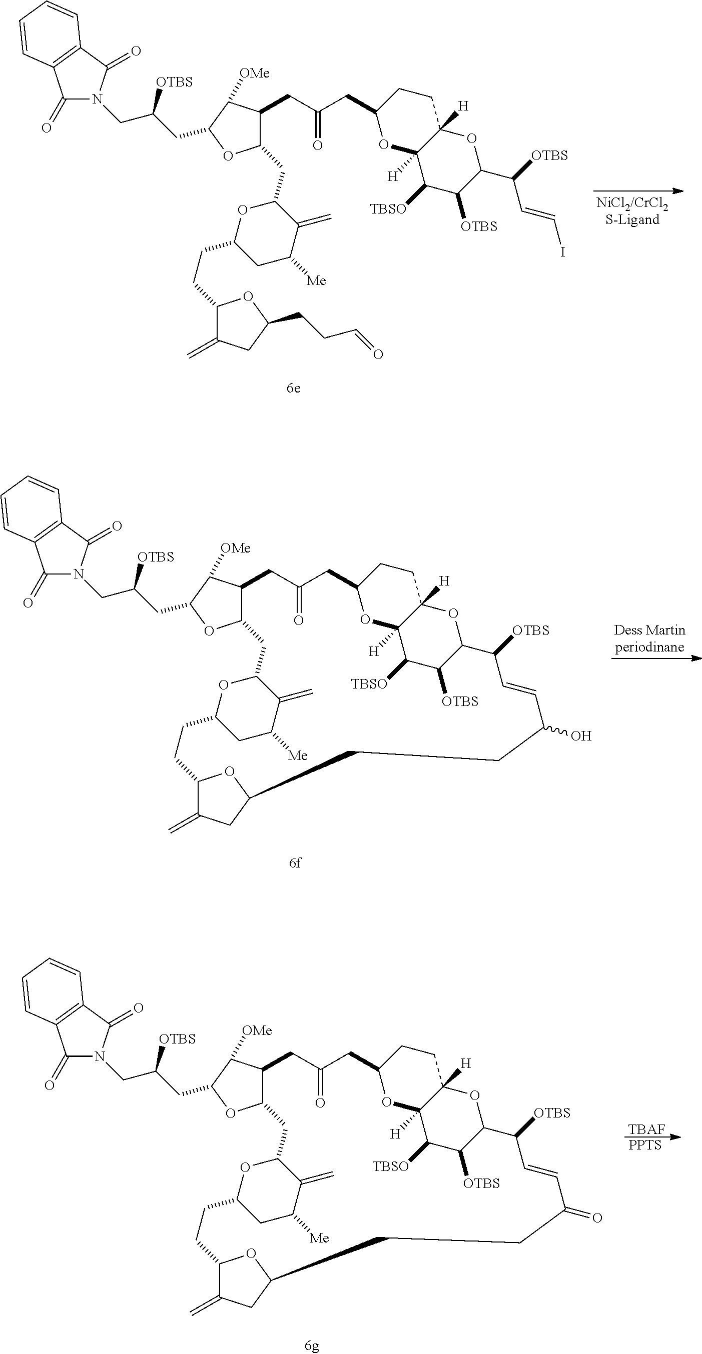

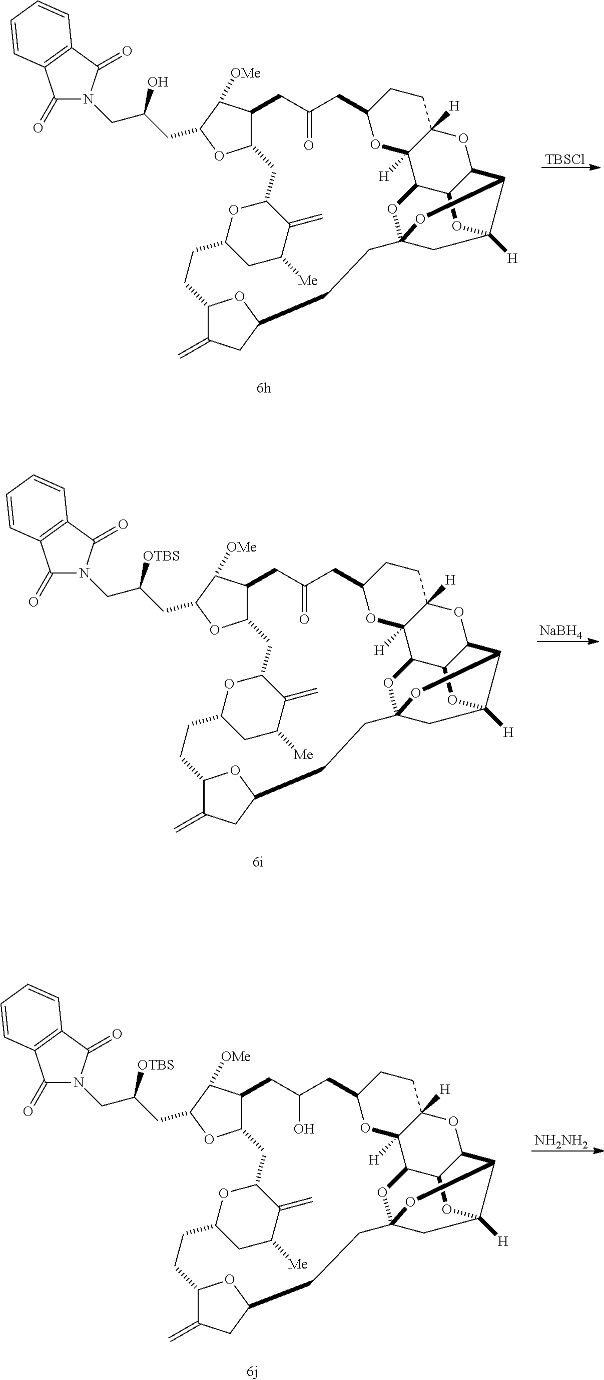

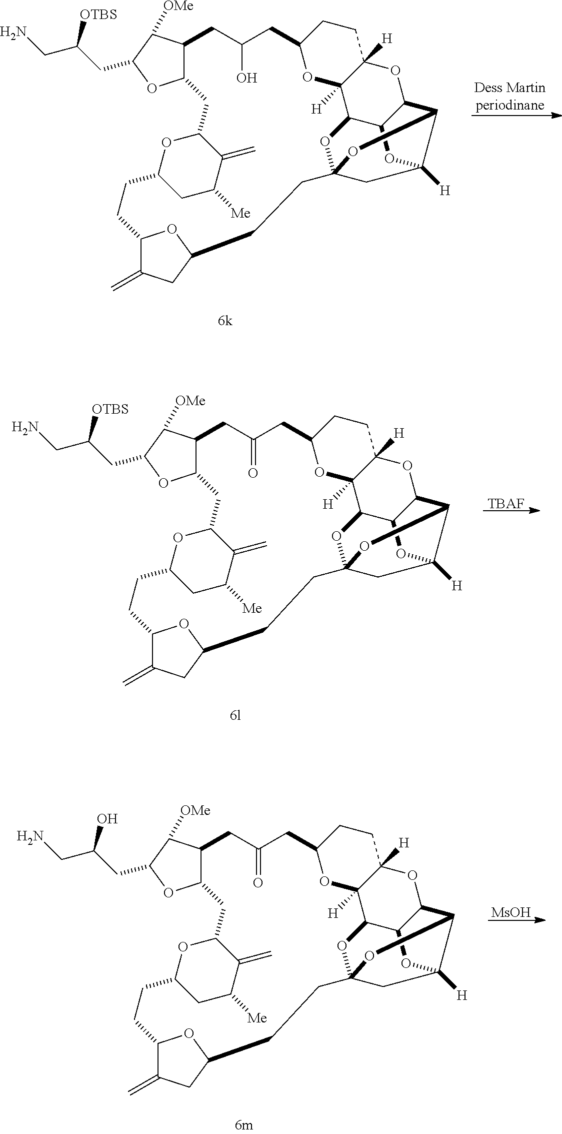

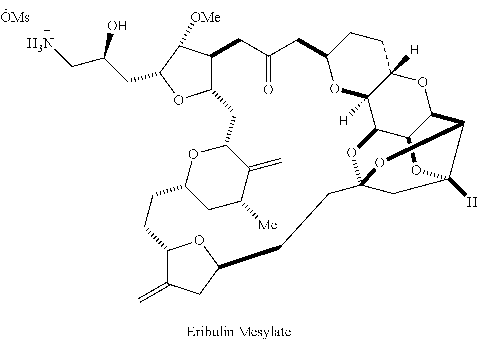

### Scheme 1 Examples and Experiments

### EXAMPLE 1

### Preparation of Tosyl 5a

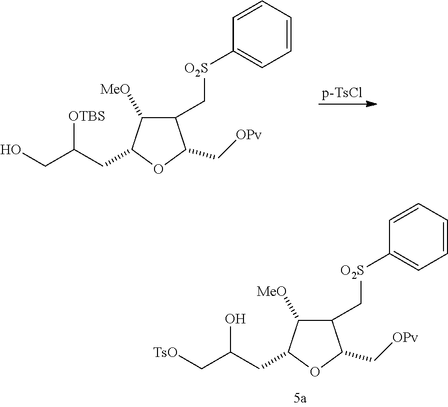

Diol (10 g) is dissolved in CH₂CI₂ (50 ml) and the resulting solution is cooled to 0° C. To the solution of diol is added pyridine (5.0 eq., 0.13 g), catalytic DMAP and TsCI (1.0 eq., 4.28 g) at 0° C. The reaction mixture is allowed to slowly warm to room temperature and is stirred at room temperature until TLC analysis (1:1—heptanes:EtOAc) indicates the reaction to be complete. The reaction is quenched with sat. aq. NH₄CI (5 v). The organic layer is separated and washed once more with sat. aq. NH₄CI, followed by 1M HCI. The organic layer is dried over Na₂SO₄, filtered and concentrated in vacuum. The crude product is purified by column chromatography (SiO₂, 3:1-1:1 heptanes:EtOAc) to obtain the product 5a.

### EXAMPLE 2

### Preparation of Phthalimide 5b

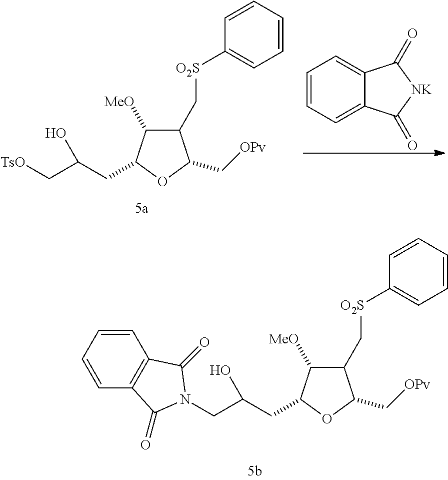

Tosyl derivative 5a (10.0 g) is dissolved in DMF (50 ml) and to this solution is added potassium phthalimide (3.0 eq. 9.27 g) at room temperature. The reaction mixture is stirred at room temperature until TLC analysis indicates that the starting material is consumed. The reaction mixture is quenched with water, diluted with diethyl ether and the layers are separated. The aqueous layer is further extracted with diethyl ether and the combined organics are dried over Na₂SO₄, filtered and concentrated in vacuum. The crude product is purified by column chromatography (SiO₂, 1:0-1:1 heptane:EtOAc) to afford product 5b.

### EXAMPLE 3

### Preparation of Silyl Ether 5c

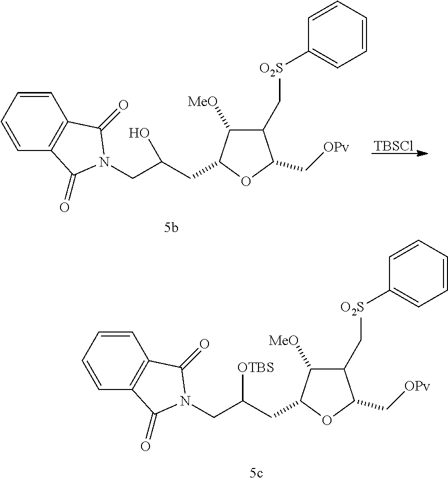

Imidazole (21 g, 308 mmol) and TBSCl (26.5 g, 176 mmol) were added to a solution of phthalimide 5b (25.2 g, 44 mmol) in DMF (90 mL) at room temperature. After 18 h, the reaction mixture was diluted with saturated aqueous NaHCO₃ (250 mL), stirred for 1 h and extracted with CH₂Cl₂ (3×100 mL). The combined organic layers were dried over Na₂SO₄, concentrated and purified by flash chromatography (5% EtOAc-hexanes) to afford silyl ether Sc (17.6 g).

### EXAMPLE 4

### Preparation of Alcohol 5d

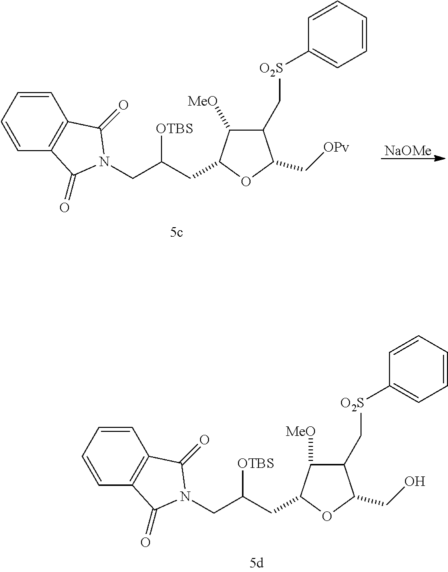

Sodium methoxide (25% in MeOH, 0.474 gm, 8.77 mmol) was added to a solution of silyl ether 5c (10.3 g, 15 mmol) in MeOH (150 ml) at 25 to 30° C. After 18 hrs., the reaction was quenched with ammonium chloride solution (150 mL) and extracted with ethyl acetate (150 mL×3). The combined organic layers were dried over Na₂SO₄, concentrated and purified by flash chromatography (20% EtOAc-hexanes) to afford alcohol 5d (9.07 g).

### EXAMPLE 5

### Preparation of Aldehyde 5e

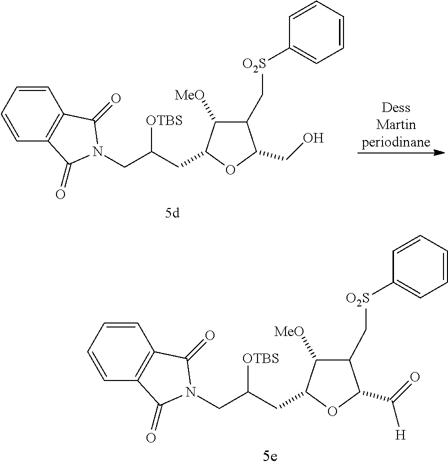 [SMILES: CO[C@@H]1C(CS(=O)(=O)c2ccccc2)[C@H](CO)O[C@@H]1CC(CN1C(=O)c2ccccc2C1=O)O[Si](C)(C)C(C)(C)C ; "DessMartinperiodinane" ; CO[C@@H]1C(CS(=O)(=O)c2ccccc2)[C@H](C=O)O[C@@H]1CC(CN1C(=O)c2ccccc2C1=O)O[Si](C)(C)C(C)(C)C]

The alcohol 5d (0.94 g, 1.57 mmol, 1.0 eq.) was dissolved in dichloromethane (16 mL) at room temperature. Dess Martin periodinane (1.66 g, 3.92 mmol, 2.5 eq) was added in one portion and the reaction mixture was stirred for 1.5 hours. The reaction was quenched by the addition of saturated aqueous sodium bicarbonate solution (75 mL) and 10% (w/w) sodium thiosulfate solution (75 mL) and further diluted with MTBE (50 mL). The resulting mixture was stirred for 60 min, diluted with brine (15 mL) and the layers were separated. The aqueous phase was further extracted with MTBE (2×30 mL) and the combined organic layers were dried over MgSO₄, filtered and concentrated. The crude product was purified by column chromatography using a Biotage Isolera, 100 g Snap Ultra column and 5-10% acetone in dichloromethane as an eluent. The product 5e was afforded as a white foam (0.75 g).

### EXAMPLE 6

### Preparation of Olefin 5f

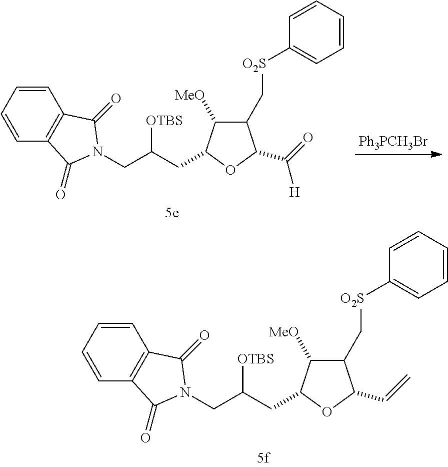

n-BuLi (1.6 M, 20 mL, 30 mmol) was added dropwise to a solution of CH₃PPh₃Br (10.1 g, 30 mmol) in THF (350 mL) and DMSO (100 mL) at 0° C. After 1 hr., a solution of the crude aldehyde 5e in THF (50 mL) was added. The reaction mixture was warmed to room temperature and stirred for 3 hr. Saturated aqueous NH₄Cl was added and the mixture was extracted with EtOAc (3×500 mL). The combined extracts were washed with brine, dried over Na₂SO₄, concentrated and purified by flash chromatography (7% EtOAc-hexanes) to afford olefin 5f (12.6 g).

### EXAMPLE 7

### Preparation of Alcohol 5g

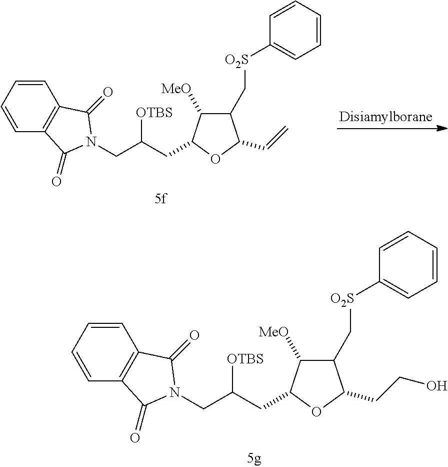 [SMILES: C=C[C@@H]1O[C@H](CC(CN2C(=O)c3ccccc3C2=O)O[Si](C)(C)C(C)(C)C)[C@H](OC)C1CS(=O)(=O)c1ccccc1 ; "Disiamylborane" ; CO[C@@H]1C(CS(=O)(=O)c2ccccc2)[C@H](CCO)O[C@@H]1CC(CN1C(=O)c2ccccc2C1=O)O[Si](C)(C)C(C)(C)C]

The solution of 2-methyl-2-butene (1.76 mL) in THF (7.4 mL) was added dropwise to a 0° C. cooled solution of 1M borane-THF complex solution (8.8 mL) and stirred for 2hrs. The prepared cold solution was added dropwise to solution of olefin 5f (1.50 g) in THF (3.0 mL) at 0° C. and stirred overnight. The reaction mass was cooled to −10° C. and added water (3.0 mL). 10% NaOH (8.8 mL) and then 30% H₂O₂ (8.8 mL) solution was added at −10° C. to 0° C. and stirred for 2 hrs. The aqueous solution was extracted with EtOAc (3×10.5 mL). The combined extracts were washed with brine, dried over Na₂SO₄, concentrated and purified by flash chromatography (10% EtOAc-hexanes) to afford alcohol 5g (0.64 g).

### EXAMPLE 8

### Preparation of Aldehyde (F-5)

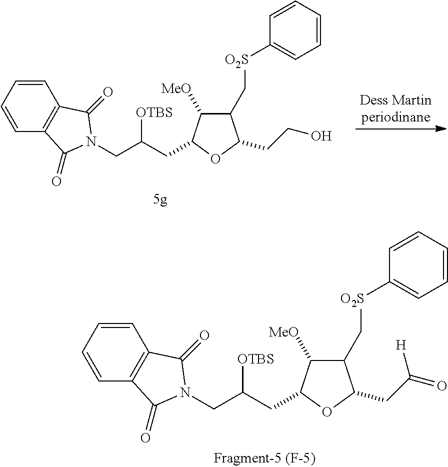 [SMILES: CO[C@@H]1C(CS(=O)(=O)c2ccccc2)[C@H](CCO)O[C@@H]1CC(CN1C(=O)c2ccccc2C1=O)O[Si](C)(C)C(C)(C)C ; "Dess Martinperiodinane" ; CO[C@@H]1C(CS(=O)(=O)c2ccccc2)[C@H](CC=O)O[C@@H]1CC(CN1C(=O)c2ccccc2C1=O)O[Si](C)(C)C(C)(C)C]

The alcohol 5g (0.96 g, 1.57 mmol, 1.0 eq.) was dissolved in dichloromethane (16 mL) at room temperature. Dess Martin periodinane (1.66 g, 3.92 mmol, 2.5 q) was added in one portion and the reaction mixture was stirred for 1.5 hours. The reaction was quenched by the addition of saturated aqueous sodium bicarbonate solution (75 mL) and 10% (w/w) sodium thiosulfate solution (75 mL) and further diluted with MTBE (50 mL). The resulting mixture was stirred for 60 min, diluted with brine (15 mL) and the layers were separated. The aqueous phase was further extracted with MTBE (2×30 mL) and the combined organic layers were dried over MgSO₄, filtered and concentrated. The crude product was purified by column chromatography using a Biotage Isolera, 100 g Snap Ultra column and 5-10% acetone in dichloromethane as an eluent. The product F-5 was afforded as a white foam (0.76 g).

### Scheme 2 Examples and Experiments

### EXAMPLE 9

### Preparation of Pivalate 6a

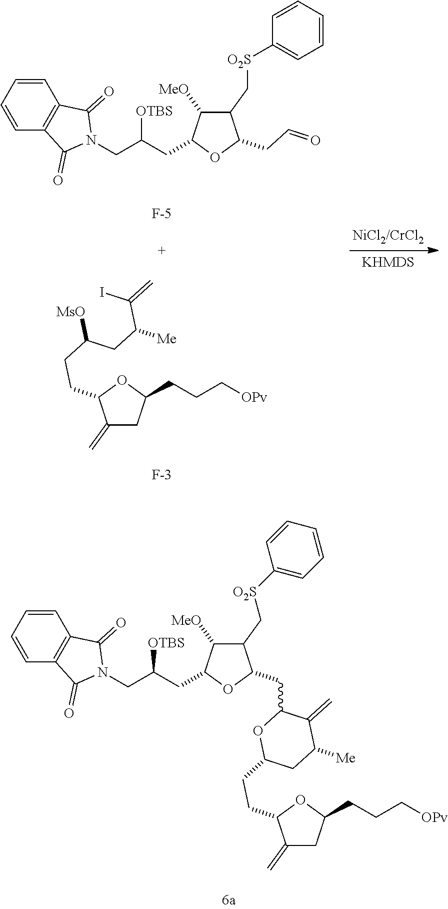

0.1% NiCl₂/CrCl₂ (w/w, 3.21 g) and 1% NiCl₂/CrCl₂ (w/w, 4.31 g) were added to a solution of aldehyde F-5 (3.85 g, 6.25 mmol), key fragment vinyl iodide F-3 (5.10 g, 9.16 mmol), THF (85 mL) and DMF (21 mL) at room temperature in a glove box. The reaction mixture was stirred for 24 hrs., removed from the glove box, cooled to 0° C., diluted with EtOAc (100 mL), quenched with saturated NH₄Cl (200 mL) and stirred for 30 mins. The separated aqueous phase was extracted with EtOAc (6×170 mL) and the combined organic layers were dried over Na₂SO₄, concentrated and purified by column chromatography. The fractions evaporated and compound (4.61 g) was taken without purification for further processing. The intermediate (4.61 g, 4.48 mmol,) was dissolved in THF (150 mL), cooled to 0° C. and treated with KHMDS (0.5 M in toluene, 14 mL, 7.0 mmol) over a 2 min. period. After stirring at 0° C. for 15 min, the reaction was quenched with saturated aqueous NH₄Cl (150 mL) and warmed to room temperature. The reaction mixture was then extracted with MTBE and several times with dichloromethane. The filtrate was concentrated and subsequently purified by column chromatography using a Biotage Isolera, 100 g Snap column and 5-10% acetone in dichloromethane as eluent. The product pivalate 6a was afforded as a foam (4.21 g).

### EXAMPLE 10

### Preparation of Alcohol 6b

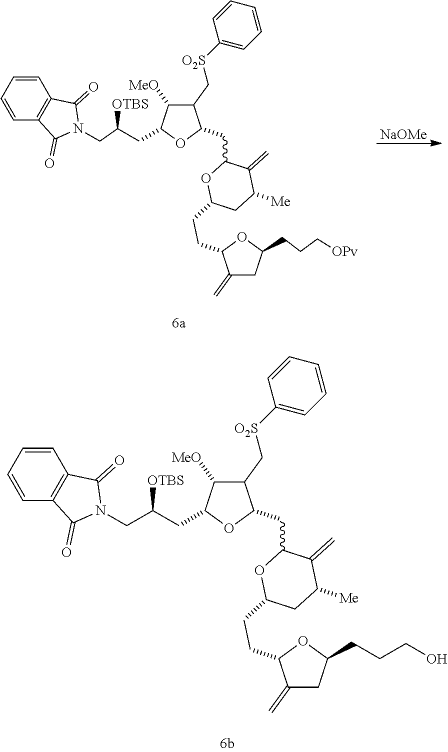

Sodium methoxide (25% in MeOH, 0.046 gm, 0.868 mmol) was added to a solution of pivaloyl 6a (1.65 g, 1.74 mmol) in MeOH (82.5 ml) at 25 to 30° C. After 24 hrs., the reaction was quenched with ammonium chloride solution (82.5 mL) and extracted with ethyl acetate (82.5 mL×3). The combined organic layers were dried over Na₂SO₄, concentrated and purified by flash chromatography (20% to 40% EtOAc-hexanes) to give alcohol 6b (1.38 g) as a residue.

### EXAMPLE 11

### Preparation of Sulfone Diol 6c

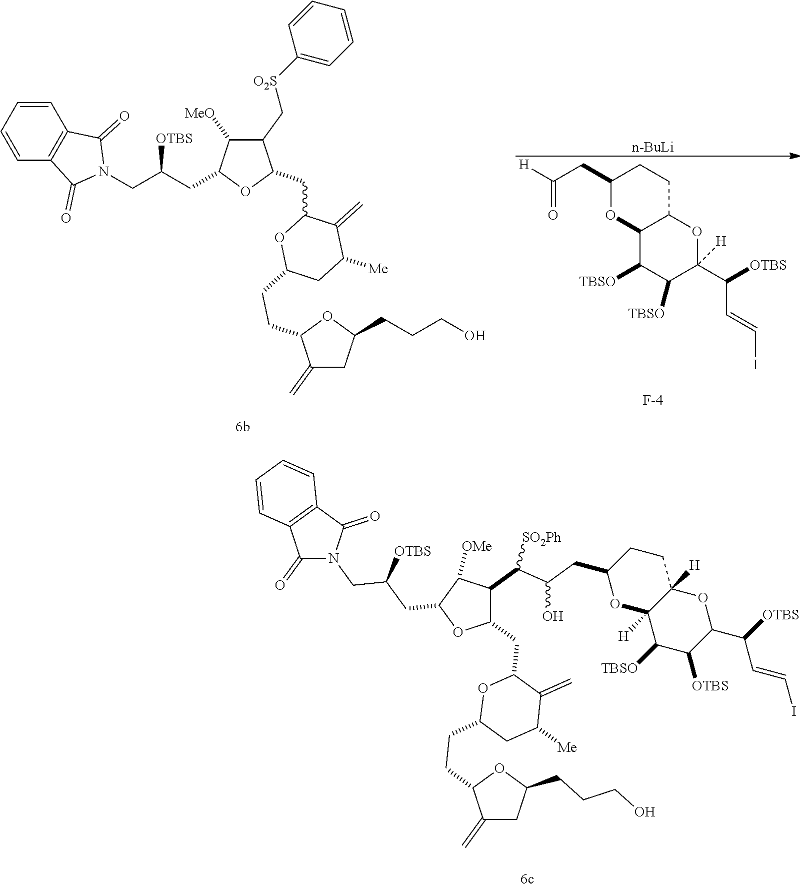

The alcohol 6b (1.10 g, 1.28 mmol, 1.0 eq.) was dissolved in THF (13 mL) and the solution was cooled to 0° C. n-BuLi (1.4M in hexane) was added dropwise until the bright yellow colour of the sulfone anion was just visible and persisted (1.12 mL) and a second aliquot of n-BuLi (0.91 mL, 1.28 mmol, 1.0 eq) was then added to the reaction mixture. The resulting yellow solution was stirred at 0° C. for 10 min and then cooled to −70° C. Compound 4a (1.42 g, 1.92 mmol, 1.5 eq) was dissolved in hexanes (20 mL) and added to the reaction mixture, which was stirred at −70° C. for an additional 45 min. The cooling bath was removed and reaction was quenched with the addition of saturated aqueous ammonium chloride solution (20 mL) and the resulting mixture was extracted with MTBE (3×20 mL). The combined organic layers were washed with brine (20 mL), dried over Na₂SO₄, filtered and concentrated. The crude material was purified by column to give sulfone diol 6c.

### EXAMPLE 12

### Preparation of Keto Sulfone 6d

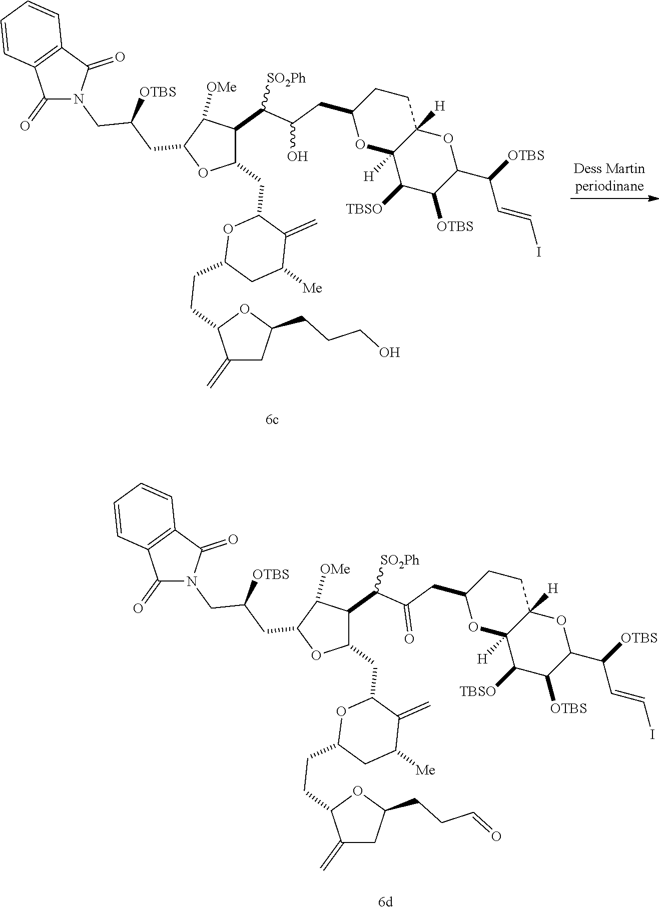

The sulfone diol 6d (2.52 g, 1.57 mmol, 1.0 eq.) was dissolved in dichloromethane (16 mL) at room temperature. Dess Martin periodinane (1.66 g, 3.92 mmol, 2.5 q) was added in one portion and the reaction mixture was stirred for 1.5 hours. The reaction was quenched by the addition of saturated aqueous sodium bicarbonate solution (75 mL) and 10% (w/w) sodium thiosulfate solution (75 mL) and further diluted with MTBE (50 mL). The resulting mixture was stirred for 60 min, diluted with brine (15 mL) and the layers were separated. The aqueous phase was further extracted with MTBE (2×30 mL) and the combined organic layers were dried over MgSO₄, filtered and concentrated. The crude product was purified by column chromatography using a Biotage Isolera, 100 g Snap Ultra column and 5-10% acetone in dichloromethane as an eluent. The Keto sulfone 6d was afforded as a foam (1.69 g).

### EXAMPLE 13

### Preparation of Ketone 6e

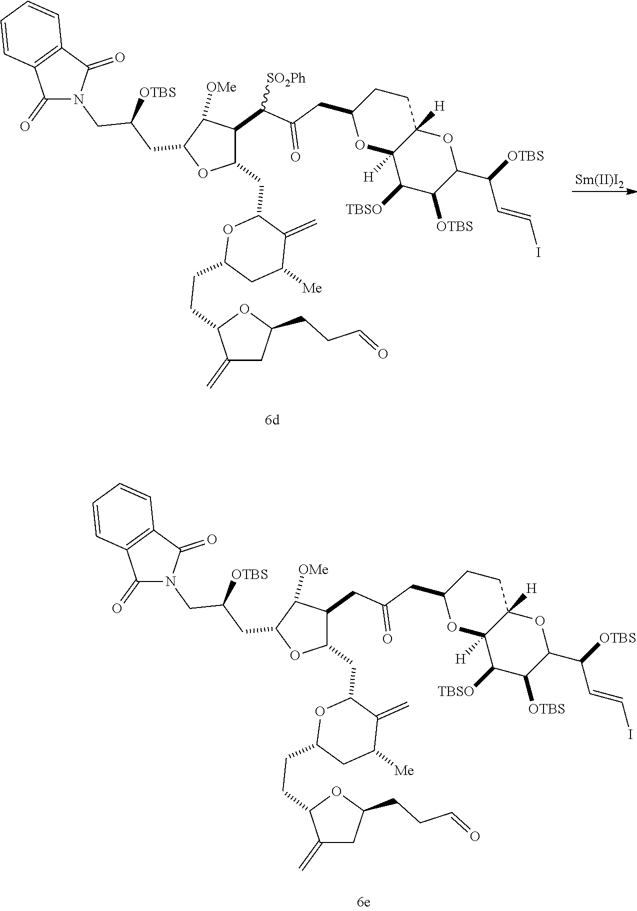

The solution of keto sulfone 6d (0.03 mL) in THF was added to a solution of the sulfone in THF at −78° C. After 5 minutes, additional SmI₂ reagent, 0.05 mL, was added. After a few additional minutes, more reagent, 0.25 mL, was added. The cooling bath was removed and saturated aqueous sodium bicarbonate (3 mL) was added. The mixture was partitioned between ether and water and the usual work-up gave ketone 6e (9.0 mg) of an oil.

### EXAMPLE 14

### Preparation of Allylic Alcohol 6f

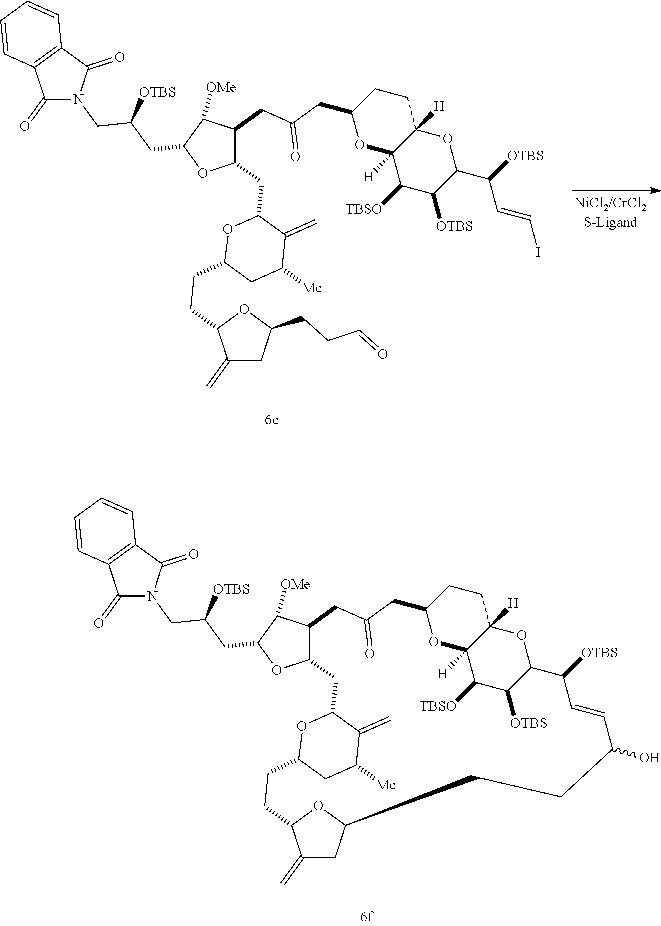

In a glove box, NiCl₂/CrCl₂ (1% w/w, 1.09 g, 8.86 mmol) was added to a solution of ketone 6e (1.02 g, 0.70 mmol) in THF (60 mL) and DMF (15 mL) at room temperature. After stirring for 2 days the reaction mixture was taken out of the glove box, cooled to 0° C., quenched with saturated aqueous NH₄Cl (30 mL) and stirred at 0° C. for 20 min. After addition of H₂O (10 mL), the two layers were separated and the aqueous layer was extracted with EtOAc (5×60 mL). The combined organic phases were washed with brine, dried over Na₂SO₄, concentrated and purified by column chromatography (15% EtOAc-hexanes) to furnish a mixture of epimers (0.82 g) as a solid foam. Although the epimers could be separated by prep TLC (20% EtOAc-hexanes), they were carried forward as a mixture.

### EXAMPLE 15

### Preparation of Enone 6g

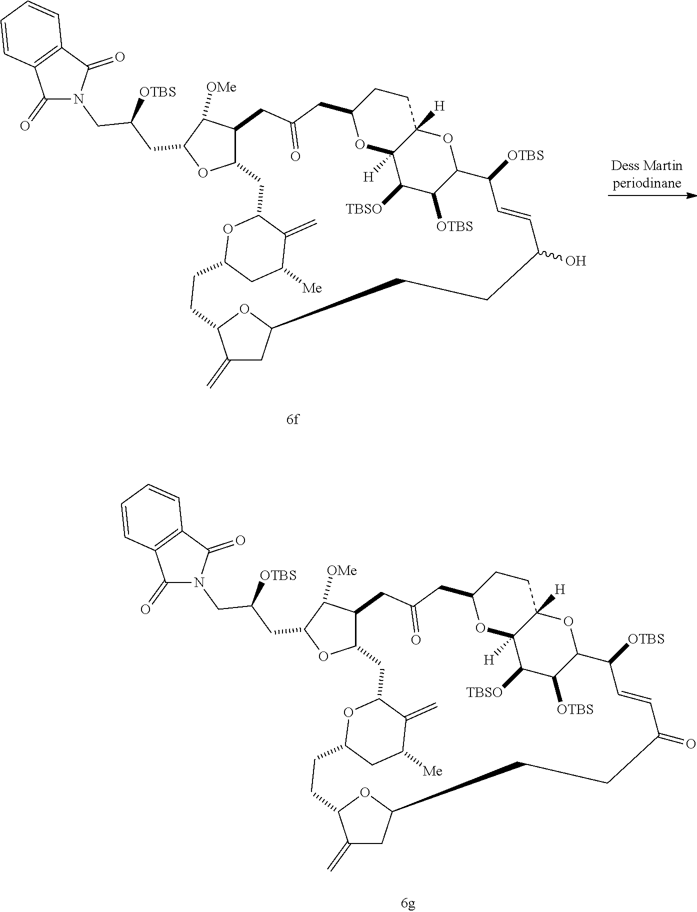

A mixture of allylic alcohol (0.80 g, 0.60 mmol) and Dess-Martin periodinane (0.26 g, 0.60 mmol) in CH₂Cl₂ (30 mL) was stirred for 30 mins. at room temperature. Additional Dess-Martin periodinane (0.26 g, 0.60 mmol) was added to the mixture and stirring was continued for an additional 1.5 hrs. The mixture was then diluted with Et₂O (100 mL), stirred for 15 min and filtered through Celite. The filtrate was washed with saturated aqueous NaHCO₃ (100 mL) and the separated aqueous layer was extracted with Et₂O (3×). The combined organic phases were dried over Na₂SO₄, concentrated and purified by column chromatography (10% to 15% EtOAc-hexanes) to give enone 6g (0.63 g) as a residue.

### EXAMPLE 16

### Preparation of Dione 6h

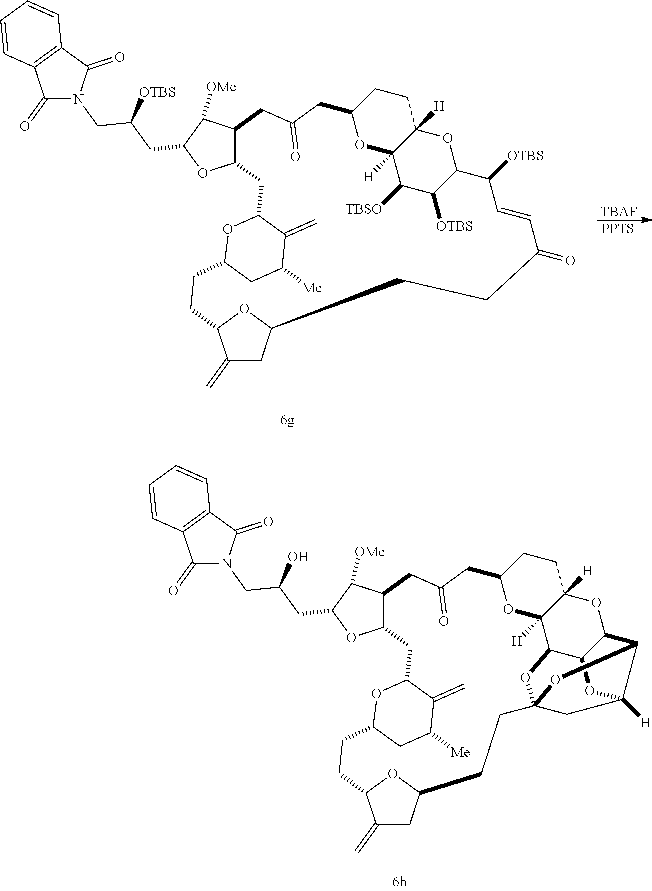

TBAF (1 M in THF containing 0.5 M imidazole HCl, 4.60 mL, 4.60 mmol) was added over 2 mins. to a solution of enone (0.64 g, 0.48 mmol,) in THF (29 mL) at room temperature and the resulting mixture was stirred for 18 hrs. After dilution with hexanes (10 mL), the reaction mixture was directly loaded onto a SiO₂ column packed with 50% EtOAc-hexanes and eluted with 50% EtOAc-hexanes (1 L) followed by 10% MeOH/EtOAc to collect a mixture of intermediates. After solvent removal, the residue was dissolved in CH₂Cl₂ (15 mL) and treated with PPTS (645 mg). After stirring for 1 hr. at room temperature, additional PPTS (414 mg) was added and the resulting white suspension was stirred for 4.5 hrs. The reaction mixture was then directly loaded onto a SiO₂ column packed with 70% EtOAc-hexanes and eluted with 70% EtOAc/hexanes (0.5 L), EtOAc (1 L). Elution with 5% to 10% MeOH/EtOAc furnished pure dione (181 mg) and elution with 15% MeOH-EtOAc gave additional semi-pure product, which after purification by preparative TLC (10% MeOH-EtOAc) provided additional pure dione (40 mg). The dione (total 221 mg) was obtained as a solid.

### EXAMPLE 17

### Preparation of Silyl Ether 6i

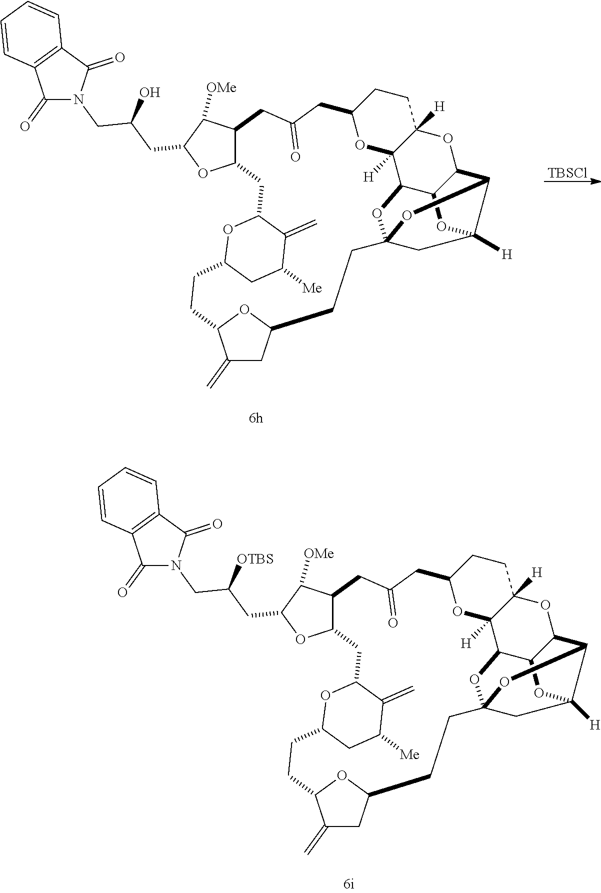

Imidazole (21 g, 308 mmol) and TBSCl (26.5 g, 176 mmol) were added to a solution of hydroxyl compound (37.8 g, 44 mmol) in DMF (90 mL) at room temperature. After 18 hrs, the reaction mixture was diluted with saturated aqueous NaHCO₃ (250 mL), stirred for 1 hr. and extracted with CH₂Cl₂ (3×100 mL). The combined organic layers were dried over Na₂SO₄, concentrated and purified by flash chromatography (5% EtOAc-hexanes) to afford silyl ether 6i (30.4 g).

### EXAMPLE 18

### Preparation of Alcohol 6j

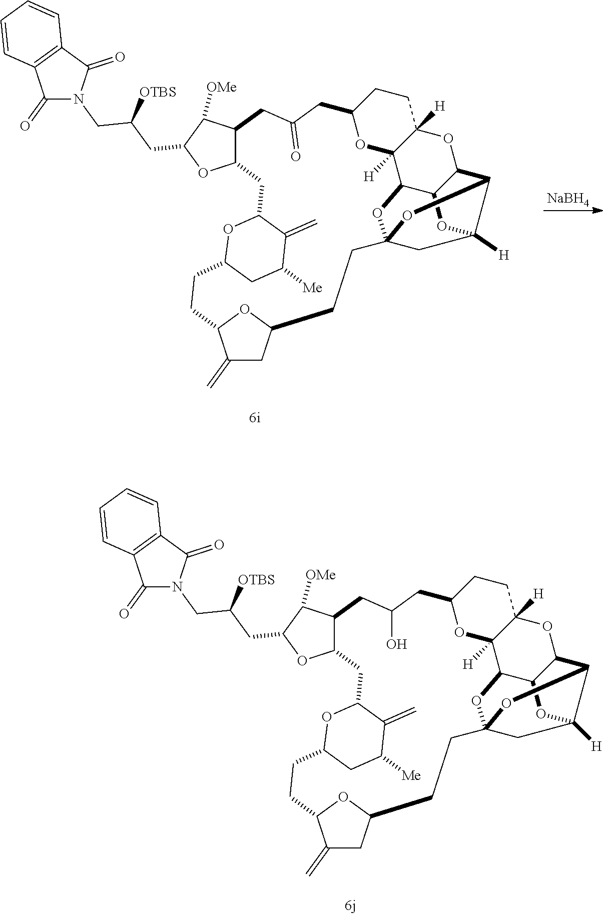

To a solution of silyl ether 6i (82.8 g, 85 mmol) in MeOH (200 mL), NaBH₄ was added portion-wise at 0° C. The progress of the reaction was monitored by TLC analysis. When the starting material had been essentially consumed, the solvent was evaporated under reduced pressure. The residue was re-dissolved in EtOAc (150 mL), washed with saturated NH₄Cl solution (200 mL), brine (50 mL), and dried over Na₂SO₄. After evaporating the solvent, the crude alcohol 6j was used without further purification.

### EXAMPLE 19

### Preparation of Amino-Alcohol 6k

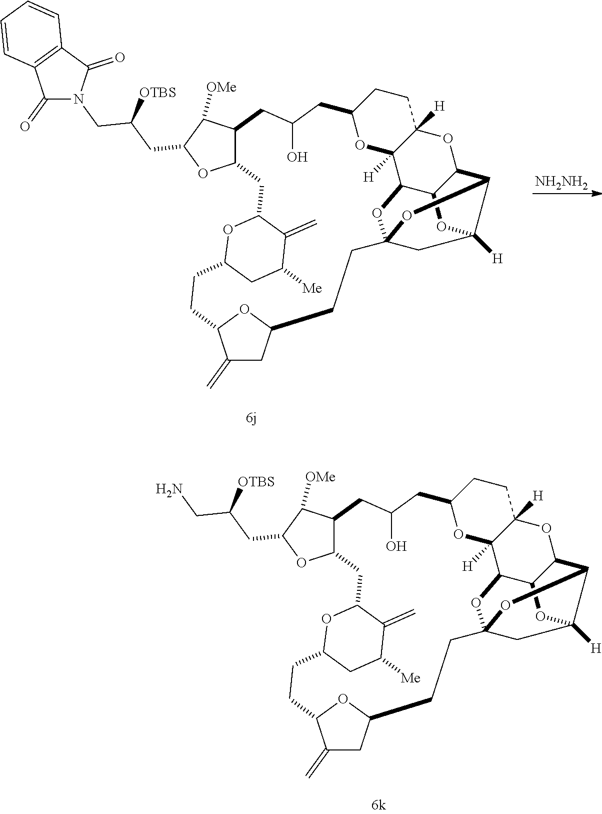

To a solution of alcohol 6j (1.75 g, 1 eq.) in 100 mL THF was added 0.34 ml (6 eq.) hydrazine and the reaction mixture was stirred at room temperature for 3 days, then 18 mL 10% NaOH solution was added and the reaction mixture stirred an additional 2 days, then reduced to 30 mL for purification by preparative HPLC.

### EXAMPLE 20

### Preparation of Amino-Ketone 6l

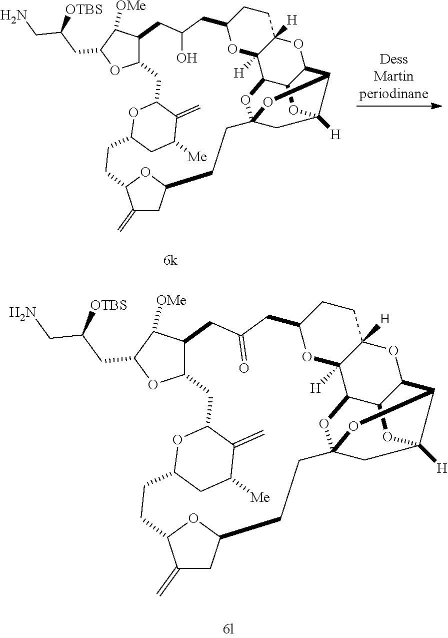

Amino-alcohol 6k (1.32 g, 1.57 mmol, 1.0 eq.) was dissolved in dichloromethane (16 mL) at room temperature. Dess Martin periodinane (1.66 g, 3.92 mmol, 2.5 q) was added in one portion and the reaction mixture was stirred for 1.5 hours. The reaction was quenched by the addition of saturated aqueous sodium bicarbonate solution (75 mL) and 10% (w/w) sodium thiosulfate solution (75 mL) and further diluted with MTBE (50 mL). The resulting mixture was stirred for 60 min, diluted with brine (15 mL) and the layers were separated. The aqueous phase was further extracted with MTBE (2×30 mL) and the combined organic layers were dried over MgSO₄, filtered and concentrated. The crude product was purified by column chromatography using a Biotage Isolera, 100 g Snap Ultra column and 5-10% acetone in dichloromethane as an eluent. The product amino-ketone 6k was afforded as a foam (1.06 g).

### EXAMPLE 22

### Preparation of Eribulin Free Base 6m

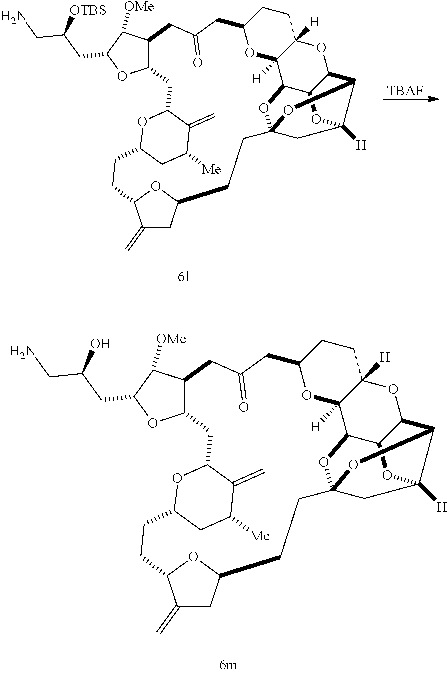

TBAF (1M in THF containing 0.5 M imidazole HCl, 4.60 mL, 4.60 mmol) was added over 2 mins. to a solution of amino-ketone 6l (0.41 g, 0.48 mmol,) in THF (29 mL) at room temperature and the resulting mixture was stirred for 18 hrs. After dilution with hexanes (10 mL), the reaction mixture was directly loaded onto a SiO₂ column packed with 50% EtOAc-hexanes and eluted with 50% EtOAc-hexanes (1L) followed by 10% MeOH/EtOAc to collect fractions of intermediates.

### EXAMPLE 23

### Preparation of Eribulin Mesylate

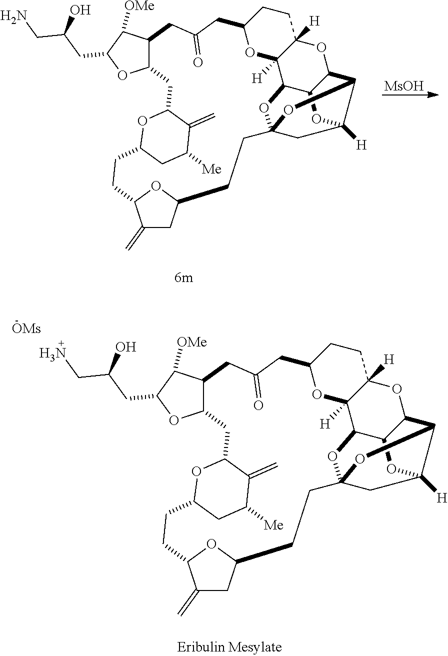

Eribulin free base (4.67 g) was dissolved in acetonitrile (59.1 mL) and water (3.1 mL) and treated with a solution of methanesulfonic acid (MsOH, 0.41 mL) and NH₄OH (18.7 mL) in acetonitrile (62.4 mL). The mixture was concentrated in vacuum at 24° C. or below and azeotroped repeatedly with anhydrous acetonitrile (23.4 mL) in vacuum at 24° C. or below to remove water. The residue was dissolved in 75% v/v anhydrous dichloromethane in n-pentane (110 mL) and filtered. The filtrate was concentrated in vacuum at 24° C. or below. The residue was dissolved in 50% v/v anhydrous dichloromethane in n-pentane (116 mL), and the solution was transferred through a filter to anhydrous pentane (0.326 kg) in the separate reactor. The resulting precipitate was stirred for 29 hours. The precipitates were filtered, washed with n-pentane (0.292 kg), and dried under nitrogen flow in vacuum until the residual solvent levels reached the target numbers: n-pentane≦25000 ppm; 2-methylbutane≦1000 ppm; 2,2-dimethylbutane≦1000 ppm; and cyclopentane≦1000 ppm. After drying, the precipitates were mixed in vacuum to give eribulin mesylate (4.59 g). The drug substance was filled in a polytetrafluoroethylene (PTFE) bottle. The PTFE bottle was packed in an aluminum laminate bag.

Although the compounds, schemes and methods of the present disclosure have been described with reference to exemplary embodiments thereof, the present disclosure is not limited thereby. Indeed, the exemplary embodiments are implementations of the disclosed methods are provided for illustrative and non-limitative purposes. Changes, modifications, enhancements and/or refinements to the disclosed methods may be made without departing from the spirit or scope of the present disclosure. Accordingly, such changes, modifications, enhancements and/or refinements are encompassed within the scope of the present invention. All publications, patent applications, patents, figures and other references mentioned herein are expressly incorporated by reference in their entirety.

What is claimed is:

1. A method of making eribulin mesylate having the formula I 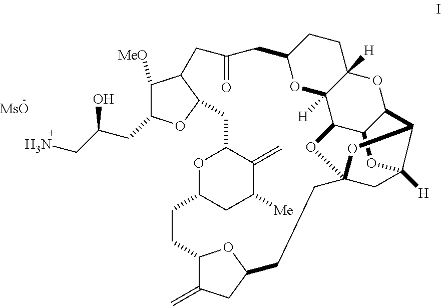
comprising the steps of:
a. reacting a compound of the formula F-5 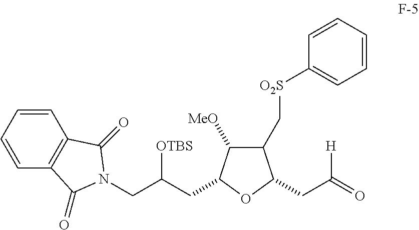
with a compound of the formula F3 in the presence of NiCl₂/CrCl₂ 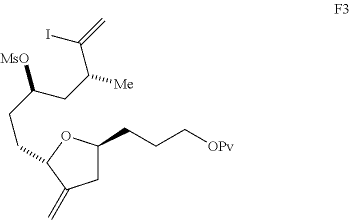
to obtain a compound of the formula 6a 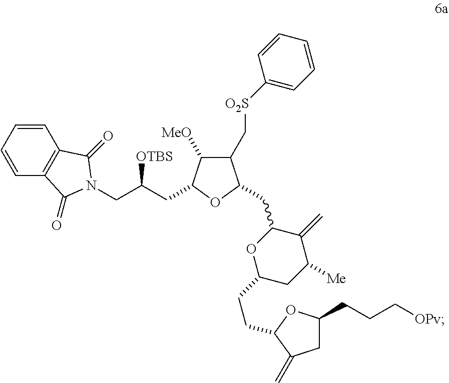
b. reacting the compound of formula 6a with sodium methoxide to obtain a compound of the formula 6b 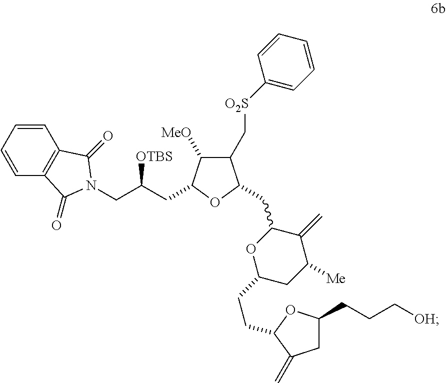
c. reacting a compound of the formula 6b with compound of the formula F4 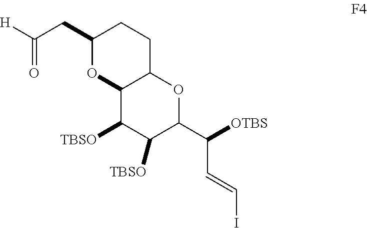
to obtain a compound of the formula 6c 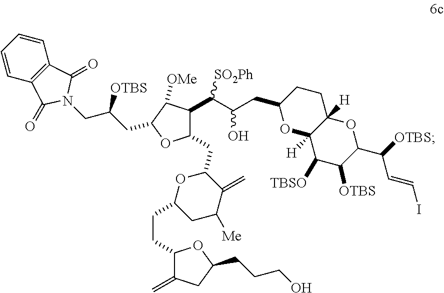
d. oxidizing a compound of the formula 6c to obtain a compound of the formula 6d 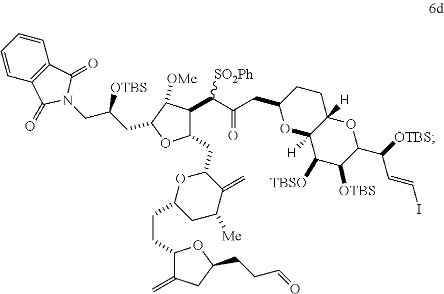
e. reducing the arylsulfonyl moiety of the compound of formula 6d by reacting a compound of the formula 6d with a reducing agent to produce a compound of the formula 6e 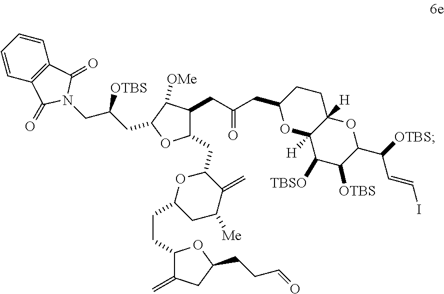
f. subjecting the compound of the formula 6e to an intramolecular coupling reaction to produce a compound of the formula 6f 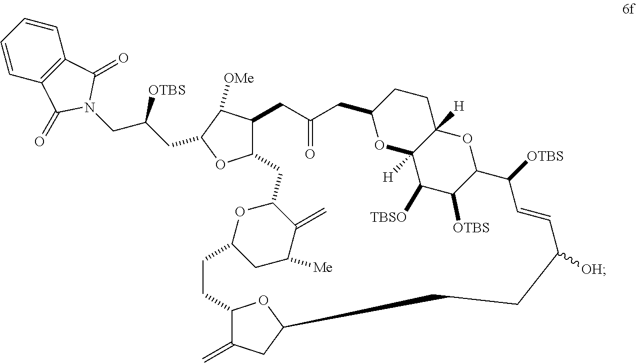
g. oxidizing the compound of the formula 6f to produce a compound of the formula 6g 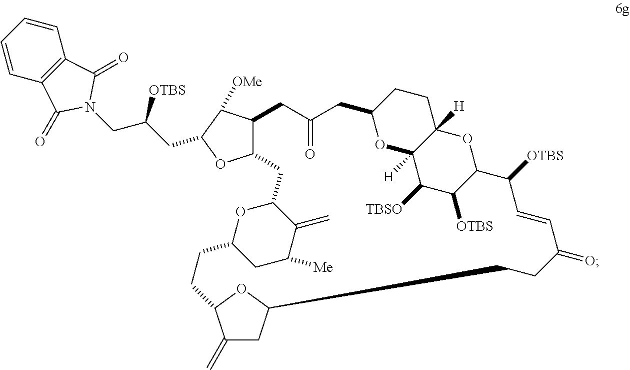
h. desilylating and subjecting to an intramolecular cyclization reaction the compound of the formula 6g to produce a compound of the formula 6h 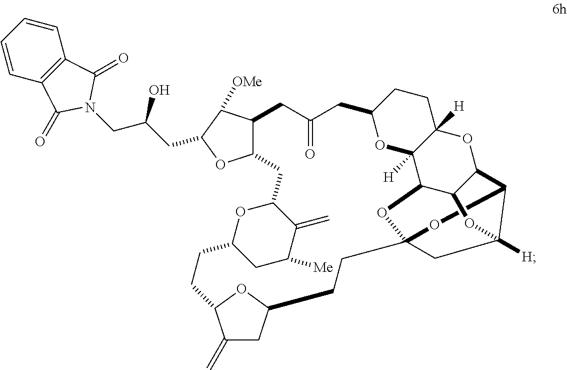
i. reacting the compound of the formula 6h with a tert-butyldimethylsilyl and a base to produce a compound of the formula 6i 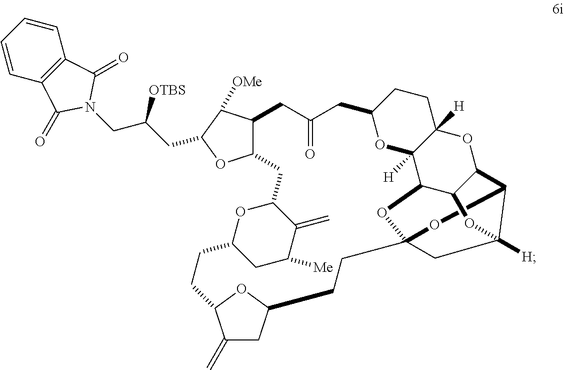
j. oxidizing the compound of the formula 6i to produce a compound of the formula 6j 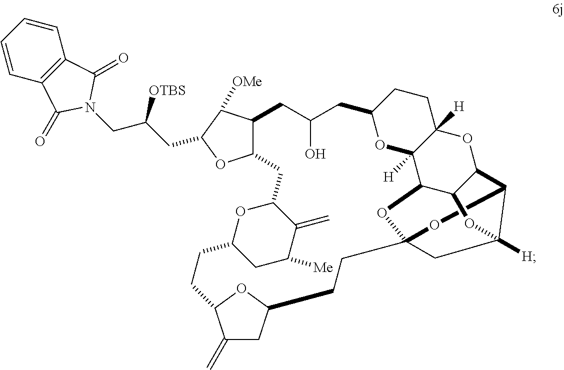
k. reducing the phthalimide group of the compound of the formula 6j to produce a compound of the formula 6k 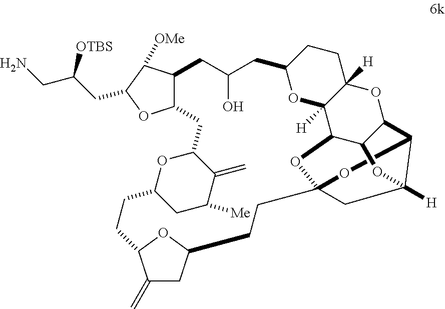
l. oxidizing a compound of the formula 6k to produce compound of the formula 6l 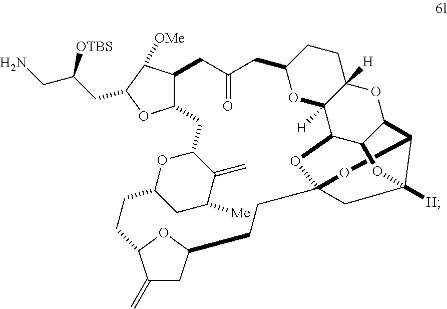
m. desilylating a compound of the formula 6l to produce a compound of the formula 6m 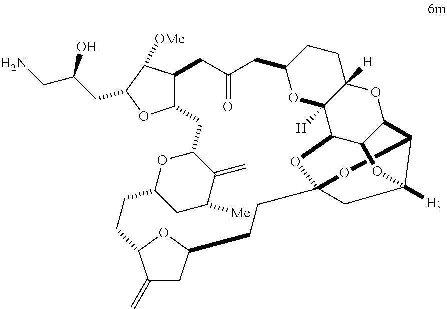
and
n. treating a compound of the formula 6m with methanesulfonic acid to produce eribulin mesylate.

2. The method according to claim 1 wherein at least one of the oxidizing steps is carried out using Dess-Martin periodinane.

3. The method according to claim 1 wherein the step of reducing the arylsulfonyl moiety is carried out using hydrazine.

4. The method according to claim 1 wherein the step of subjecting the compound of the formula 6e to an intramolecular coupling reaction is carried out using a NiCl₂/CrCl₂ catalyst.

5. The method according to claim 1 wherein the desilylation step is carried out using a fluoride containing reagent and the intramolecular cyclization reaction is carried out using pyridinium p-toluenesulfonate (PPTS).

6. A method of making eribulin mesylate comprising the steps of:
a. reacting a compound of the formula F-5 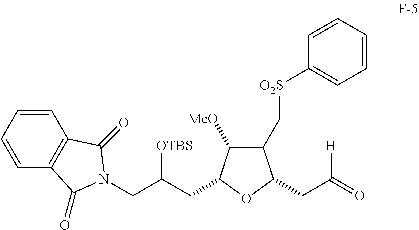
with a compound of the formula F3 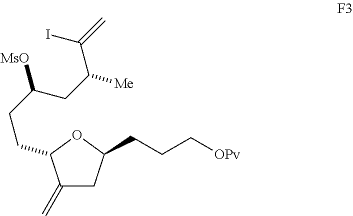
to obtain a compound of the formula 6a 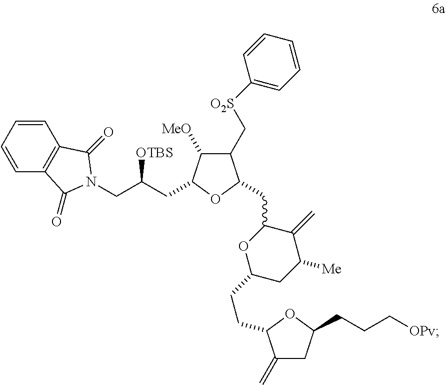
b. reacting the compound of formula 6a with sodium methoxide to obtain a compound of the formula 6b 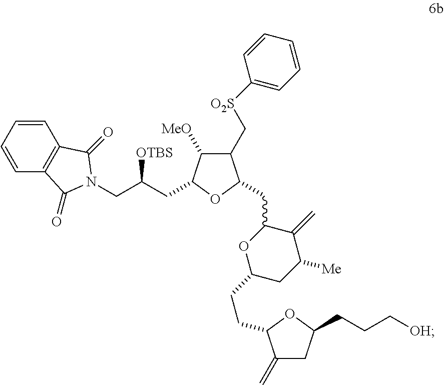
c. oxidizing the compound of formula 6b to produce a compound of the formula 7c 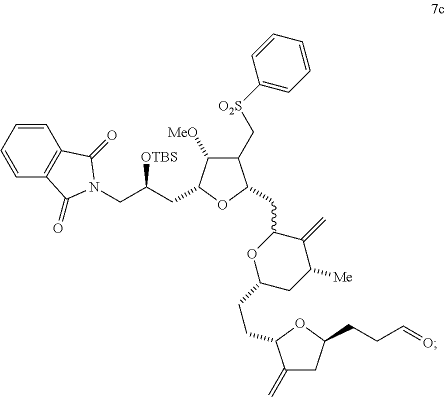
d. reacting a compound of the formula 7c with a compound of the formula F4 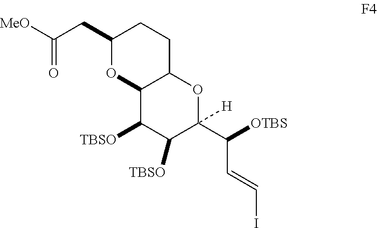
to produce a compound of the formula 7d 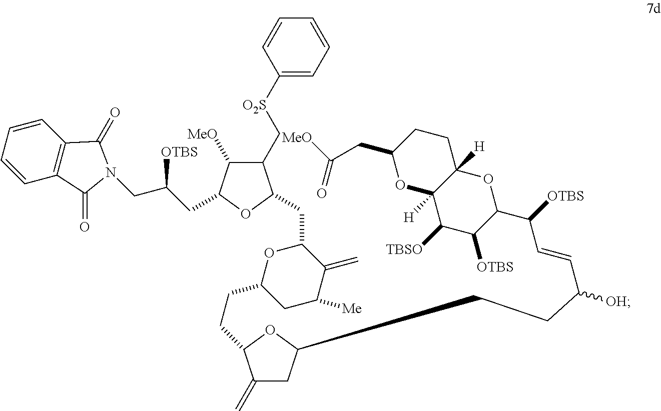
e. oxidizing the compound of formula 7d to produce a compound of the formula 7e 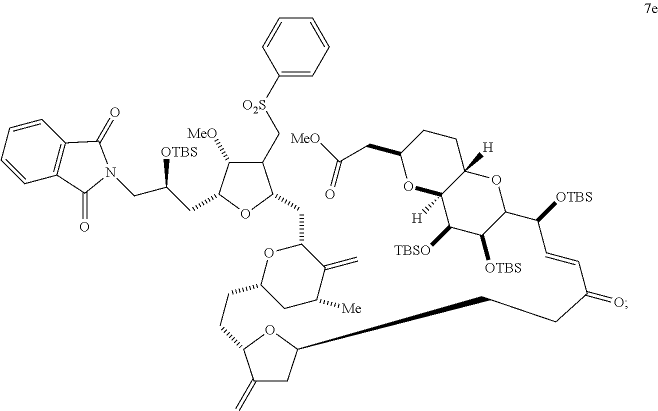
f. desilylating and subjecting to an intramolecular cyclization reaction the compound of the formula 7e to produce a compound of the formula 7f 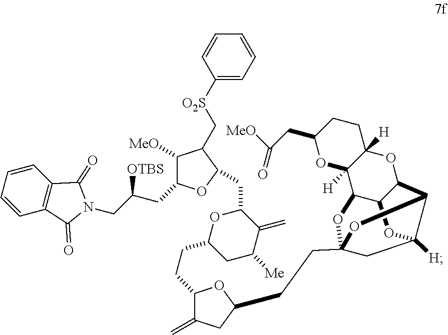
g. subjecting the compound of the formula 7f to a reducing agent to produce a compound of the formula 7g 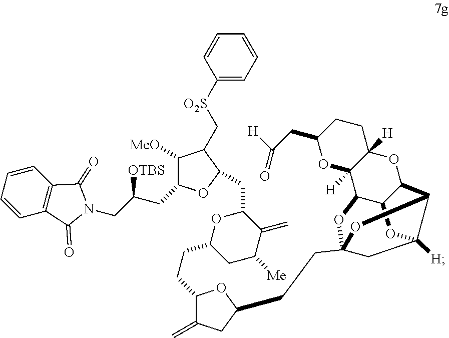
h. conducting an intramolecular coupling reaction of the compound of formula 7g to produce a compound of the formula 7h 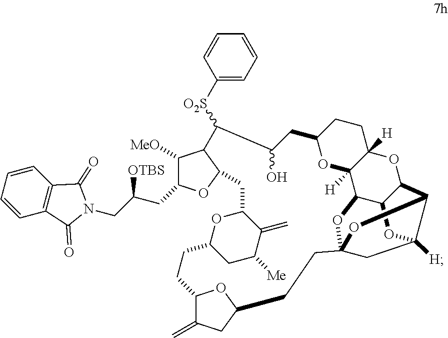
i. oxidizing the compound of the formula 7h to produce a compound of the formula 7i; 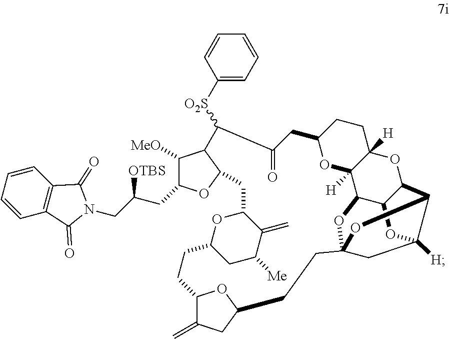
j. reducing the arylsulfonyl moiety of the compound of the formula 7i to obtain a compound of the formula 6i 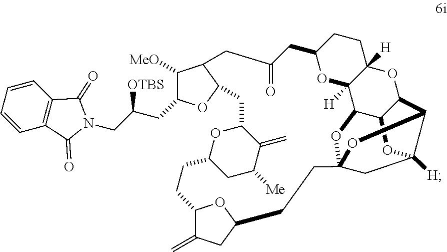
k. subjecting the compound of the formula 6i to a reducing agent to obtain a compound of the formula 6j; 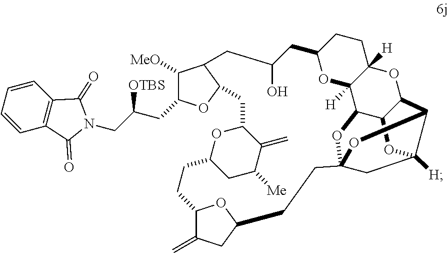
l. treating the compound of the formula 6j with a reducing agent operable to reduce the phthalimide group of the compound of formula 6j to obtain a compound of the formula 6k 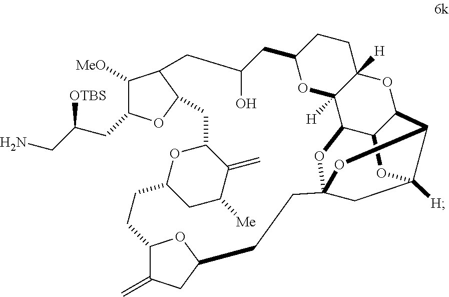
m. oxidizing the compound of the formula 6k to obtain a compound of the formula 6l 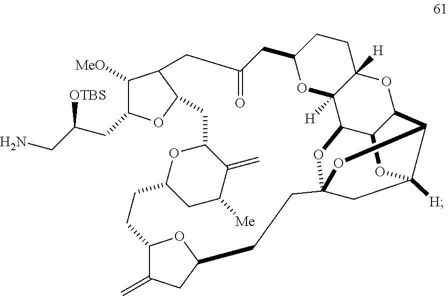
n. deprotecting the compound of the formula 6l to obtain a compound of the formula 6m 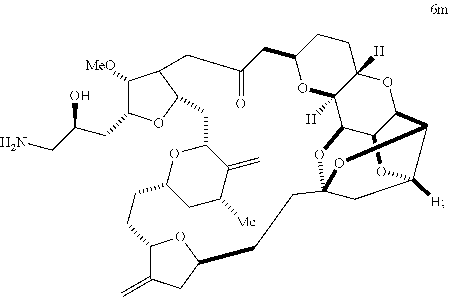
o. treating the compound of the formula 6m with methanesulfonic acid to obtain eribulin mesylate.

7. The method according to claim 6 wherein at least one of the oxidizing steps is carried out using Dess-Martin periodinane.

8. The method according to claim 6 wherein the step of reducing the arylsulfonyl moiety of the compound of the formula 7i is carried out using hydrazine.

9. The method according to claim 6 wherein the step of conducting an intramolecular coupling reaction of the compound of formula 7g is carried out using a NiCl₂/CrCl₂ catalyst.

10. The method according to claim 6 wherein the step of desilylating the compound of the formula 7e is carried out using a fluoride containing reagent and the intramolecular cyclization reaction is carried out using pyridinium p-toluenesulfonate (PPTS).

11. A method of making a compound of the formula F-5 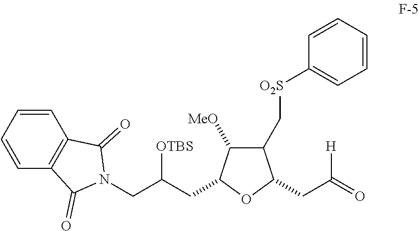
comprising the steps of:
a. reacting a compound of the formula 5a with potassium phthalimide to produce a compound of the formula 5b 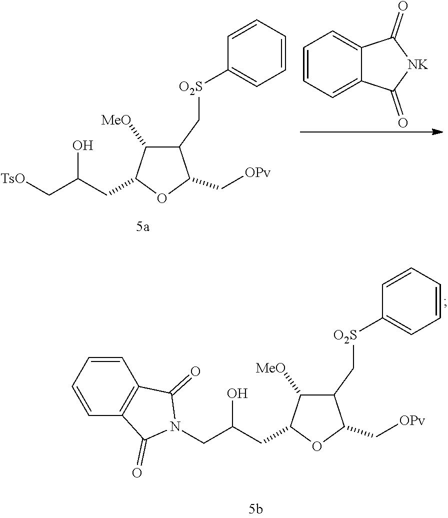
b. protecting of the alcohol group of the compound of formula 5b to produce a compound of the formula 5c 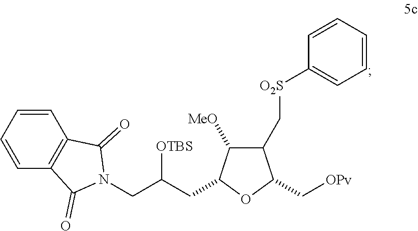
c. reacting the compound of formula 5c with sodium methoxide to produce a compound of the formula 5d 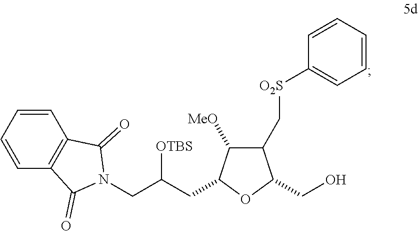
d. oxidizing the compound of formula 5d to produce a compound of the formula 5e 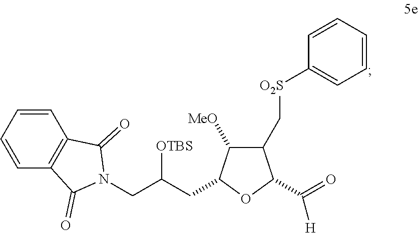
e. converting the ketone functional group of the compound of formula 5e to an alkene to produce compound of the formula 5f 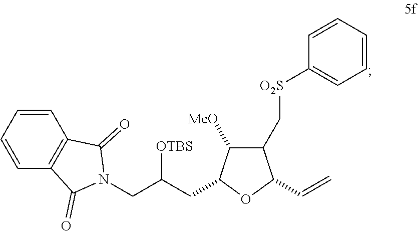
f. subjecting the compound of 5f to a hydroboration-oxidation reaction to produce a compound of the formula 5g 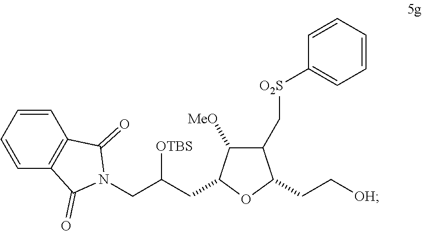
and
g. oxidizing the compound of the formula 5g to produce the compound of the formula F-5.

12. The method according to claim 11 wherein at least one of the oxidation steps is carried out using Dess-Martin periodinane.

13. The method according to claim 11 wherein the step of converting the ketone functional group of the compound of formula 5e to an alkene is conducted using CH₃PPh₃Br.

14. The method according to claim 11 wherein the hydroboration-oxidation reaction is carried using disiamylborane.
